(19) United States
(12) Patent Application Publication
(10) Pub. No.: US 2025/0169742 A1
(43) Pub. Date: May 29, 2025

(54) METHOD AND DEVICE FOR IMPROVED CLINICAL VESTIBULAR TESTING

(71) Applicant: UNIVERSITY OF MISSISSIPPI MEDICAL CENTER, Jackson, MS (US)

(72) Inventors: Wu ZHOU, Jackson, MS (US); Hong ZHU, Jackson, MS (US)

(21) Appl. No.: 18/842,760

(22) PCT Filed: Mar. 2, 2023
(86) PCT No.: PCT/US2023/014399

§ 371 Date: Aug. 29, 2024

Related U.S. Application Data
(60) Provisional application No. 63/315,651, filed on Mar. 2, 2022.

(51) Int. Cl. A61B 5/395; A61B 5/397

(52) U.S. Cl. CPC A61B 5/395; A61B 5/397

(57) ABSTRACT

A method of discriminative vestibular-evoked myogenic potential testing (VEMP) in a subject, comprising a first and second acoustic stimuli to the subject to selectively activate otolith afferents or to activate both canal and otolith afferents to provide a response.

### GOVERNMENT INTEREST

This invention was made with government support under grant numbers R01DC012060 and R01DC008585 awarded by the National Institutes of Health (NIH). The government has certain rights in the invention.

### TECHNICAL FIELD

The presently-disclosed subject matter generally relates to methods and devices for improved clinical vestibular testing. More specifically, the presently-disclosed subject matter relates to methods and devices for discriminate VEMP testing between the otolith and canal afferents.

### BACKGROUND AND SUMMARY

Millions of people have experienced some form of vestibular dysfunction. This can include dizziness, balance issues, and vertigo. Diagnosis of vestibular dysfunction remains difficult. Various methods for stimulating vestibular organs for the purpose of testing and diagnosing vestibular dysfunction, restoring vestibular sensory inputs, or providing vestibular afferent signals to downstream neural circuits are known in the art. However, many of these methods are expensive, invasive, and for diagnostic tests can require a high level of expertise to perform and properly interpret. Accordingly, there are a number of problems in the art relating to vestibular dysfunction that can be addressed.

One method is a vestibular evoked myogenic potential (VEMP) test. The purpose of the VEMP test is to determine if the peripheral vestibular end organs are intact and working properly. When functioning correctly, vestibular peripheral end organs send signals to the muscles of the eyes and the neck to stabilize gaze and head during movements. Good balance and vision rely on functional vestibular reflexes.

During a cervical VEMP test it is common to recline at an angle and have surface electrodes attached to the subject's neck muscles. Then the subject will listen to a loud sound while lifting his or her head up slightly. The electrodes will measure the responses from the subject's vestibular system, and a symmetrical response from each ear can be measured. The VEMP can help localize the side that may be involved in causing dizziness or imbalance.

While VEMP tests are routinely used to test otolith function, the specific vestibular afferent neurons and central circuits activated by auditory frequency VEMP stimuli remains unclear.

The present invention adds a novel approach to the VEMP test that allows for the first time correct isolation of the otolith function, and isolation of the otolith and canal function. This allows for a more accurate picture of the vestibular system, enabling a care provider to identify the cause of dizziness, balance issues, or vertigo.

Inner ear vestibular end organs function to detect head acceleration and orientation with respect to gravity and send sensory signals to the CNS to maintain stability of gaze, posture and blood pressure during movement (Goldberg et al., 2012). In addition to being activated by physiological stimuli (i.e., head rotation, translation and tilt), vestibular sensory organs can also be activated by non-physiological stimuli including galvanic current (Dlugaiczyk et al., 2019), infrared heat (Rajguru et al., 2011), magnetic force (Ward et al., 2019) and loud sounds (Young et al., 1977). These stimuli offer novel approaches to assess vestibular function. Among them, acoustic activation of the vestibular system has been widely adopted in clinics to test otolith function. Acoustic activation of vestibular organs was first observed in pigeons with fenestrated bony canals (Tullio, 1929), sensitivity arising from introduction of a compliant window in the bony labyrinth (Minor et al., 1998; Iversen et al., 2018, Greiser et al., 2016). But, sensitivity to acoustic sound and bone conducted vibrations is not restricted to pathological conditions. Loud sounds can evoke vestibular responses in healthy human subjects (Parker et al., 1978) and in animals with intact labyrinths (Young et al., 1977, in squirrel monkeys; Wit et al., 1984, in pigeons; McCue and Guinan, 1994a, 1994b, 1995, 1997, in cats; Curthoys and Vulovic, 2011, Murofushi et al., 1995, in guinea pig; Carey et al., 2004, in chinchilla; Zhou et al., 2003, 2007, Xu et al., 2009, in monkeys; Zhu et al., 2011, 2014, in rats).

Activation of vestibular afferents by acoustic stimuli leads to compensatory motor outputs that can be observed clinically, most commonly by measuring click-evoked electromyographic potentials from the tonically contracted sternocleidomastoideoles (SCM) (i.e. the cervical VEMP, cVEMP) (Colebatch and Halmagyi, 1992) or the extraocular muscles (i.e., the ocular VEMP, oVEMP) (Jombik and Bahyl, 2005; Todd et al., 2007). The cVEMP and oVEMP are mediated by the vestibulo-collic reflex (VCR) pathways and the vestibulo-ocular (VOR) pathways, respectively (Wilson and Schor, 1999; Uchino et al; 2005; Uchino and Kushiro, 2011). Vestibular afferent neurons with calyx synaptic endings contacting Type I hair cells are the most sensitive to auditory frequency stimuli (Curthoys et al., 2016; Curthoys et al., 2017). VEMPs are now an important part of the neuro-otological test battery for characterizing a variety of vestibulopathies, including superior canal dehiscence (SCD), vestibular neuritis, Meniere's disease and vestibular schwannoma (for reviews, Colebatch, 2001; Goldberg et al., 2012).

From a clinical perspective, it is generally thought that cVEMPs and oVEMPs test saccular function and utricular function, respectively (Curthoys, 2010). But, there is evidence that sound also activates the semicircular canals (Goldberg et al., 2012; Young et al., 1977; Carey et al., 2004; Zhou et al., 2007; Xu et al., 2009; Zhu et al., 2011, 2014), making it difficult to precisely interpret sound-evoked motor outputs driven by the neural circuits that receive inputs from more than one vestibular organ. The relative activation of individual vestibular afferent neurons innervating the semicircular canal organs and the otolith organs in response to tone bursts delivered at specific frequencies and intensities is quantified in this application. Results demonstrate that sound-evoked vestibular inputs to the CNS depend on specifics of the sound stimulus, the vestibular organ of origin, and the afferent type. Findings also provide guidelines for design of stimuli to preferentially activate vestibular otolith organs, and provide a substrate for interpretation of vestibular inputs driving VEMP outputs.

One embodiment of the present invention is a method of discriminative vestibular-evoked myogenic potential testing (VEMP) in a subject. This embodiment comprises providing a first acoustic stimuli to the subject that activates otolith afferents to provide a response, this first acoustic stimuli being at a first predetermined frequency and a first set predetermined intensities. It also includes providing a second acoustic stimuli to the subject to activate both canal and otolith afferents to provide a response, this second acoustic stimuli being at a second predetermined frequency and a second set of predetermined intensities. It also includes determining and comparing the responses of the first and second acoustic stimuli to assess canal and otolith contributions to sound-evoked responses of the vestibular end organs.

Embodiments of the present provide a first successful process to discriminately activate, detect, and measure otolith afferents. This provides many advantages to a care provider when treating or developing a treatment plan for a patient.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-F show representative horizontal canal (HC) afferent's responses to tone bursts (125-4000 Hz, 10 ms, 60-80 dB SL, 0 dB SL is referred to the ABR threshold) delivered to the ipsilateral ear (left ear). (A) Identification of the end organ innervated by the afferent. Top trace: head velocity, positive value for rightward head rotation. Bottom trace: instantaneous firing rate of the afferent in response to horizontal rotation. The afferent had a spontaneous firing rate of 109.2 spikes/s and a CV* of 0.86. Its firing rate was increased during leftward head rotation with a gain of 1.27 spikes/s/deg/s and phase lead of 22.7 deg with respect to left head rotation (i.e., phase lag of 157.3 deg with respect to right head rotation). Vertical calibration bar is 80 deg/s for head velocity, 200 spikes/s for firing rate. (B) The afferent's raster in response to the tone bursts of 2000 Hz at 80 dB SL. (C) Peristimulus time histograms of the tone burst-evoked responses. Bin size is 0.5 ms. Vertical calibration bar is 1000 spikes/s for firing rate. (D) Spike density of the tone burst-evoked responses. Vertical calibration bar is 1000 spikes/s for firing rate. (E) Quantitative measurement of the tone-burst-evoked response. Colored traces are probability of evoking a spike (CPE) as a function of time for 2000 Hz tone bursts at three intensities (60, 70, 80 dB SL). Amplitude of evoked response is defined by CPE, which is estimated as peak probability minus the baseline probability. Latency of tone-burst evoked response is defined as the onset of the sharp increase in firing probability. (F) Tuning curves of the afferent at three intensities.

FIG. 2 shows graphs illustrating relationships between discharge regularity (CV*) and afferent responses (CPE) evoked by tone bursts of 350 Hz at different intensity levels for different end organs. Each symbol represents one afferent. AC: anterior canal units; HC: horizontal canal units; PC: posterior canal units; SO: otolith units in the superior branch (black symbols); IO: otolith units in the inferior branch (red symbols).

FIG. 3 shows graphs illustrating relationships between discharge regularity (CV*) and afferent responses (CPE) evoked by tone bursts of 1500 Hz at different intensity levels for different end organs. Each symbol represents one afferent. AC: anterior canal units; HC: horizontal canal units; PC: posterior canal units; SO: otolith units in the superior branch; IO: otolith units in the inferior branch.

FIGS. 4A-D show graphs illustrating characteristics of sound sensitive (SS) afferents in different vestibular end organs. (A) Percentages of SS afferents in regular and irregular afferents. (B) CV* of SS and NSS (not sound sensitive) afferents. (C) Spontaneous firing rates of SS and NSS afferents. (D) Head rotation gains of SS and NSS afferents. AC: anterior canal units; HC: horizontal canal units; PC: posterior canal units; SO: otolith units in the superior branch; IO: otolith units in the inferior branch. *, P<0.01; **, P<0.001; ***, P<0.0001.

FIGS. 5A-C shows graphs illustrating identification of putative calyx units by their discharge properties, i.e., relationships between 2-Hz head rotation gain and discharge regularity (CV*). (A) Anterior canal afferents (AC). (B) Horizontal canal afferents (HC). (C) Posterior canal afferents. Gray symbols are regular units, blue symbols are irregular dimorphic units and red symbols are irregular calyx units. Gains of noncalyx units related to CV* by a power law (straight lines). A curved line (a quadratic discriminant function) was used to classify the calyx units (red symbols) from the irregular dimorphic units (blue symbols). Methods adopted from Marlinski et al. (2004).

FIGS. 6A-D show graphs illustrating sound sensitivity of putative calyx units and noncalyx units. (A) Top panel, spontaneous discharge regularity of sound sensitive calyx units and noncalyx units. Lower panel, 2 Hz head rotation gain of sound sensitive calyx units and noncalyx units. (B) Top panel, spontaneous discharge regularity of putative calyx units and noncalyx units. Lower panel, 2 Hz head rotation gain of putative calyx units and noncalyx units. (C) CPEs of SS calyx units and SS noncalyx units. (D) CPEs of putative calyx units and noncalyx irregular units. AC: anterior canal units; HC: horizontal canal units; PC: posterior canal units. *, P<0.01; **, P<0.001; ***, P<0.0001.

FIGS. 7A-C show graphs illustrating frequency tuning of sound sensitive (SS) vestibular afferents to tone bursts with different frequency and intensity. (A) Averaged CPE (left panel) and percentage of SS afferents (right panel) of 80 dB SL. (B) Averaged CPE (left panel) and percentage of SS afferents (right panel) of 70 dB SL. (C) Averaged CPE (left panel) and percentage of SS afferents (right panel) of 60 dB SL. AC: anterior canal units; HC: horizontal canal units; PC: posterior canal units; SO: otolith units in the superior branch; IO: otolith units in the inferior branch.

FIGS. 8A-C show graphs illustrating frequency tuning of whole population of vestibular afferent (SS and NSS afferents) to tone bursts with different frequency and intensity. (A) Averaged CPE (left panel) and percentage of SS afferents (right panel) of 80 dB SL. (B) Averaged CPE (left panel) and percentage of SS afferents (right panel) of 70 dB SL. (C) Averaged CPE (left panel) and percentage of SS afferents (right panel) of 60 dB SL. AC: anterior canal units; HC: horizontal canal units; PC: posterior canal units; SO: otolith units in the superior branch; IO: otolith units in the inferior branch.

FIGS. 9A-E show graphs illustrating averaged peristimulus histograms of tone burst-evoked responses of different vestibular end organs. (A) Histograms of responses to tone bursts of 350 Hz at 80 dB SL (top panel); histograms of responses to tone bursts of 1500 Hz at 60 dB SL (middle panel); and summed responses to tone bursts of 350 Hz/80 dB and 1500 Hz/60 dB SL, indicating selective activation of otolith organs (bottom panel) in the AC group. (B) Histograms of responses to tone bursts of 350 Hz at 80 dB SL (top panel); histograms of responses to tone bursts of 1500 Hz at 60 dB SL (middle panel); and summed responses to tone bursts of 350 Hz/80 dB and 1500 Hz/60 dB SL, indicating selective activation of otolith organs (bottom panel) in the HC group. (C) Histograms of responses to tone bursts of 350 Hz at 80 dB SL (top panel); histograms of responses to tone bursts of 1500 Hz at 60 dB SL (middle panel); and summed responses to tone bursts of 350 Hz/80 dB and 1500 Hz/60 dB SL, indicating selective activation of otolith organs (bottom panel) in the PC group. (D) Histograms of responses to tone bursts of 350 Hz at 80 dB SL (top panel); histograms of responses to tone bursts of 1500 Hz at 60 dB SL (middle panel); and summed responses to tone bursts of 350 Hz/80 dB and 1500 Hz/60 dB SL, indicating selective activation of otolith organs (bottom panel) in the SO group. (E) Histograms of responses to tone bursts of 350 Hz at 80 dB SL (top panel); histograms of responses to tone bursts of 1500 Hz at 60 dB SL (middle panel); and summed responses to tone bursts of 350 Hz/80 dB and 1500 Hz/60 dB SL, indicating selective activation of otolith organs (bottom panel) in the IO group. AC: anterior canal units; HC: horizontal canal units; PC: posterior canal units; SO: otolith units in the superior branch; IO: otolith units in the inferior branch.

FIGS. 10A-F show distributions of tone burst-evoked action potential latencies of SS afferent neurons and latency probabilities for each group. (A-E) Distributions of tone burst-evoked action potential latencies of SS afferent neurons in the (A) AC, (B) HC, (C) PC, (D) SO, and (E) IO groups vs. CPE and CV* for 1500 Hz tone bursts at 80 dB SL. Each black dot shows the latency for and individual afferent neuron, and solid curves show the probability (P) of each latency. The probability was fit with a summation of four Gaussian curves. Phase locking of action potentials to the 1500 Hz stimulus generated horizontal bands in the scatter plots and distinct peaks in the latency histograms separated by 0.50±0.15 ms. The shortest latency corresponds to the stimulus evoking an action potential on the first cycle, while subsequent peaks correspond to action potentials evoked after skipping 1-5 cycles. (F) SO and HC afferents responded with the shortest latencies of 0.58 and 0.61 ms to the peak, while PC afferents responded with the longest latency of 1.58 ms to peak (1:0.58 ms, 2:0.28 ms, 3:1.13 ms, 4:1.58 ms). Inter-afferent variance of each peak was ˜0.03 ms.

FIGS. 11A-B show graphs illustrating idealized finite element model of the rat fluid filled labyrinth. (A) Pure tones applied to the rat bony labyrinth at the oval window (OW) predict that fluid displacement is mostly localized between the oval and round window (RW). AC: anterior canal; HC: horizontal canal; PC: posterior canal; Sacc: saccule; Utr: utricle. (B) Frequency tuning with a peak near 1500 Hz similar to experimental observations arises in the mode from the interaction of the fluid in the bony labyrinth with the round window impedance. Relative fluid velocity at the location of each vestibular end organ suggests fluid vibration is likely the primary mechanism of activation, at least for stimuli below ˜70 dB SL.

FIGS. 12A-B show graphs illustrating differential activation of canal and otolith afferents using 350 Hz and 1500 Hz tone bursts. (A) Averaged CPEs of canal afferents and otolith afferents as functions of tone intensity. Open and filled triangles are canal afferent sound sensitivity to 350 Hz and 1500 Hz tone bursts, respectively. Open and filled squares are otolith afferent sound sensitivity to 350 Hz and 1500 Hz tone bursts, respectively. Red rectangles indicate the conditions in which tone bursts only activate the otolith afferents. (B) Averaged CPEs of vestibular afferents to tone bursts of 350 Hz and 1500 Hz as functions of tone intensity. Open and filled circles are vestibular afferent sound sensitivity to tone bursts of 350 Hz and 1500 Hz, respectively. Dashed line is parallel to the regression line for the open circles.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites, and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

When open-ended terms such as “including” or ‘including, but not limited to” are used, there may be other non-enumerated members of a list that would be suitable for the making, using or sale of any embodiment thereof.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As stated above, the present invention adds a novel approach to the VEMP test that allows for the first time correct isolation of the otolith function, and isolation of the otolith and canal function. This allows for a more accurate picture of the vestibular system, enabling a care provider to identify the cause of dizziness, balance issues, or vertigo. While sound-evoked vestibular myogenic potentials (VEMPs) are widely adopted by worldwide vestibular clinics to test otolith functions, the present inventors have discovered that sound stimuli used in clinical VEMP testing not only activate the otolith afferents, but also the canals afferents. In particular, the present inventors have discovered that sound sensitivity of a vestibular afferent is affected by four factors-spontaneous firing regularity, end organ, spontaneous firing rate, and terminal type. For example, irregular calyx afferents innervating the otolith end organs with higher spontaneous firing rates tend to have larger response to sound stimulation. Further, sound sensitive canal and otolith afferents exhibited distinct properties to tone frequency.

In view thereof, the presently-disclosed subject matter includes methods for discriminative VEMPs. In some embodiments, the method includes measuring and/or evoking a response in the otolith afferents. In one embodiment, the method includes applying one or more tone bursts at a frequency below 500 Hz and an intensity of up to at least 80 dB SL. In another embodiment, the method includes applying one or more tone bursts at a frequency of 1500 Hz and an intensity of 60 dB SL or less. Alternatively, in some embodiments, the method includes measuring and/or evoking a response in both the otolith and canal afferents. In one embodiment, the method includes applying one or more tone bursts at a frequency of 1500 Hz and an intensity of at least 70 dB SL.

Without wishing to be bound by theory, it is believed that the frequencies and intensities disclosed herein may be adjusted for different subjects (e.g., rats versus humans). However, differential diagnosis of canal and otolith inputs to compensatory vestibular circuits may be made in any subject using VEMP tests combining stimuli at multiple frequencies and amplitudes. Accordingly, also provided herein, are methods of testing otolith and canal functions, the methods including using combinations of tone parameters to selectively activate otolith and canal afferents.

Also provided herein are methods of determining canal contribution to sound-evoked vestibular responses, such as VEMPs. In some embodiments, the method includes using an intensity slope ratio as an index for canal contribution. In some embodiments, the method includes generating the intensity slope ratio by plotting the sums of canal and otolith cumulative probability of evoking a spike (CPE) against tone burst intensity at different tone frequencies. For example, in one embodiment, the method includes plotting the sums of canal and otolith CPEs against tone burst intensity at two tone frequencies (e.g., 350 Hz and 1500 Hz), then determining a slope ratio based upon the plot. In another embodiment, a slop ratio of greater than 1 indicates significant contributions of the canal system, whereas a ratio of near 1 indicates lack of canal contributions or deficits in the canal system.

One embodiment of the present invention is a method of discriminative vestibular-evoked myogenic potential testing (VEMP) in a subject in need thereof, comprising providing a first acoustic stimuli to the subject to activate otolith afferents to provide a response, wherein the first acoustic stimuli is at a first predetermined frequency and at a first set predetermined intensities. The method includes a second acoustic stimuli to the subject to activate both canal and otolith afferents to provide a response, wherein the second acoustic stimuli is at a second predetermined frequency and is a second set of predetermined intensities. The responses of the first and second acoustic stimuli are determined and compared to assess canal and otolith contributions to sound-evoked responses of the vestibular end organs.

In another embodiment of the invention, the first and second acoustic stimuli are series of at least three tone bursts. These tone bursts can be generated from a state of the art VEMP device.

In another embodiment of the invention, the first predetermined frequency is at or below 500 Hz. In one aspect, the first predetermined frequency is at or below 350 Hz. In another aspect, the first predetermined frequency is any frequency between about 200 and 500 Hz.

In another embodiment of the invention, and the first set of predetermined intensities is a series of tone bursts up to about 100 dB HL. In aspects of the invention, the series of tone bursts are at least four tone bursts in intensity increments of 10 dB HL. For example, the series may comprise bursts of 60, 70, 80, and 90 dB HL. In another aspect, the series may comprises bursts of 50, 60, 70, 80, and 90 dB HL.

In another aspect the first acoustic stimuli is a series of at least four tone bursts at frequency below 500 Hz and the intensity up to about 100 dB HL. In one aspect the frequency may be between 250 and 500 Hz. The tone bursts typically are at increasing intensities up to about 100 dB HL.

Without being bound by theory or mechanism, the present inventors have discovered that the frequency at less than 500 Hz discriminately activates otolith afferents.

In an aspect of the invention, the second predetermined frequency is about 1500 Hz and the second intensity is up to 100 dB HL. For example, the second acoustic stimuli is a series of tone bursts at a frequency of about 1500 Hz and the intensity of up to about 100 dB HL. The tone bursts are at increasing intensities up to about 100 dB HL.

Similar to the first acoustic stimuli, the second acoustic stimuli may have at least four tone bursts of different intensities.

The activation of the afferents is measured by the peaks of VEMP responses. For example, the peaks of the VEMP responses are plotted to form an intensity-amplitude curve. Intensity and VEMP amplitude curves of the high frequency and low frequency are compared to obtain a ratio of the slopes of the two intensity-amplitude curves.

Another embodiment of the present invention is a method of discriminative sound-evoked vestibular myogenic potential testing (VEMP) in a subject that comprises providing a first acoustic stimuli to the subject to activate otolith afferents to provide a response, the first acoustic stimuli being at a first predetermined frequency and a first set predetermined intensities; and providing a second acoustic stimuli to the subject to activate otolith afferents to provide a response, the second acoustic stimuli being at a second predetermined frequency and a second set predetermined intensities. The responses of the first and second acoustic stimuli are determined and compared to assess selectively the otolith function or the otolith and canal function contributions to sound-evoked responses of the vestibular end organs.

Since this embodiment is related to selectively measuring otolith activity, the first predetermined frequency is below 500 Hz.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

### Example 1—Differential Activation of Canal and Otolith Afferents by Acoustic Tone Bursts in Rats

Vestibular evoked myogenic potentials (VEMPs) are routinely used to test otolith function, but which specific vestibular afferent neurons and central circuits are activated by auditory frequency VEMP stimuli remains unclear. To examine this question, the sensitivity of individual vestibular afferents in adult Sprague-Dawley rats to tone bursts delivered at 9 frequencies (125-4000 Hz) and 3 intensity levels (60, 70, 80 dB SL re: acoustic brainstem response (ABR) threshold) was examined. Afferent neuron tone sensitivity was quantified by the cumulative probability of evoking a spike (CPE). Based on a threshold CPE of 0.1, acoustic stimuli in the present study evoked responses in 78.2% (390/499) of otolith afferent neurons vs. 48.4% (431/891) of canal afferent neurons.

Organ-specific vestibular inputs to the central nervous system in response to tone bursts differ based on intensity and frequency content of the stimulus. At frequencies below 500 Hz, tone bursts primarily activated both otolith afferents, even at the highest intensity tested (80 dB SL re ABR threshold). At 1500 Hz, however, tone bursts activated the canal and otolith afferents at the moderate and high intensities tested (70, 80 dB SL), but activated only otolith afferents at the low intensity tested (60 dB SL). Within an end organ, diversity of sensitivity between individual afferent neurons correlated with spontaneous discharge rate and regularity. Examination of inner ear fluid mechanics in silico suggests that the frequency response and preferential activation of the otolith organs likely arise from inner ear fluid motion trapped near the oval and round windows. These results provide insight into understanding the mechanisms of sound activation of the vestibular system and developing novel discriminative VEMP testing protocols and interpretative guidelines in humans.

### Methods

### Animals

Ninety-nine adult Sprague-Dawley rats weighing 250-350 g (Harlan Sprague-Dawley, Indianapolis, IN, USA) were used in this study. All procedures were approved by the Institutional Animal Care and Use Committee at University of Mississippi Medical Center.

### Sound Stimulation

Tone bursts were generated by a MA3 stereo microphone amplifier (DT system, Tucker-Davis Technologies, Alachua, FL, USA). To ensure comparability of results between animals, tone intensity was referred to the threshold of the auditory brainstem response (ABR) of individual animals, which also facilitates comparison of sound intensities in animal studies to that used in the human VEMP testing (Curthoys, 2010). The ABR was measured by the methods of Simpson et al. (1985), in which stainless steel subdermal needle electrodes were placed at the vertex (active), behind the stimulated ear (reference) and in the hind leg (ground). Animals with elevated thresholds were excluded. Air-conducted tone bursts (frequency: 125-4000 Hz; duration: 10 ms (8 ms plateau, 1 ms rise/fall); polarity: rarefaction or condensation; intensity: 60, 70, 80 dB SL re ABR threshold) were randomly delivered to the left ear at a rate of 5 Hz via an insert earphone (ER-3A) and 150 trials were delivered for each condition.

### Single Vestibular Afferent Recording and Data Acquisition

Surgical procedures were performed aseptically as described before (Zhu et al., 2011, 2014). Briefly, a rat was anesthetized by sodium pentobarbital (50 mg/kg, i.p.) and maintained by injection of a dose of 5 mg/kg as needed. A surgical implanted head holder was used to stabilize the head on a stereotaxic frame (David Kopf Instruments, Tujunga, CA, USA), which was mounted on a custom-made device that can deliver head rotations in pitch, roll and yaw planes. Core body temperature was maintained at 36-37° C. with a heating pad (Frederick Haer & Company, Bowdoinham, ME, USA). The left occipital bone was opened and parts of the cerebellar hemisphere were removed to allow access of the 8th nerve by a microelectrode (10˜20 M (2) (Sutter Instruments, Novato, CA, USA) aimed at the superior or inferior branch of the vestibular nerve. Detailed single vestibular afferent recording protocols were described in earlier studies (Zhu et. al., 2011). Briefly, extracellular single vestibular fiber recording was obtained using a MNAP system (Plexon Inc., Dallas, TX, USA) with efforts to isolate and test every encountered spontaneously active nerve fiber. First, spontaneous discharge activity was recorded for about 30 seconds for calculating baseline firing rate and regularity. Second, the fiber's responses to head rotations in various planes (0.5-4 Hz with peak velocity of ˜60 degree/s) were tested to determine its vestibular end organ origin and sensitivity to rotation, reported as Gain (spike/s/deg/s) and Phase advance re: peak angular velocity (degree). Based on established criteria (Goldberg and Fernandez, 1975), vestibular afferents were classified as horizontal canal afferents (HC), anterior canal afferents (AC), posterior canal afferents (PC), superior branch otolith organ afferents (SO) and inferior branch otolith organ afferents (IO). IO afferents were saccular afferents. SO afferents, however, consist of both saccular and utricule afferents. In the present study, we did not classify SO afferents into saccular afferents and utricule afferents.

Extracellular voltage signals were recorded by a CED Power 1401 system (Cambridge Electronics Devices, Cambridge, UK) at 20 kHz with 16-bit resolution and a temporal resolution of 0.01 ms. Signals of head positions and sound trigger were sampled at 1 kHz.

### Data Analysis

Offline data analysis was performed on PC workstations using Spike 2 7.10 (Cambridge Electronics Devices, Cambridge, UK), MatLab R2020a (The MathWorks, Inc. Natick, MA, USA) and SigmaPlot 12.3 (SigmaPlot Software Inc, CA, USA).

Discharge regularity was determined by calculating normalized coefficient of variation of interspike intervals, i.e., CV*s using parameters in Lasker et al. (2008, mouse). An afferent was classified as “regular” if CV*<0.1 and “irregular” if CV*≥0.1 (Young et al., 1977; Goldberg et al., 1984). A fast Fourier transform (FFT) was performed on stimulus triggered average spike rates to calculate the gain (spike/s/deg/s) and phase (degree) relative to angular head velocity. Significance of discharge rate modulation in response to head rotation was determined by permutation analyses as described by Liu and Angelaki (2009). Briefly, a Fourier ratio (FR) (i.e., power of the fundamental frequency/power of the maximum of the first 20 harmonics) was first calculated for the binned responses (40 bins per cycle). Then, the 40 bins were shuffled randomly to destroy the modulation while maintaining its inherent variability. A FR was computed for those randomly permuted histograms for 1000 times. If the original FR was larger than 99% of the permuted data sets, the modulation was considered to be statistically significant (p<0.01).

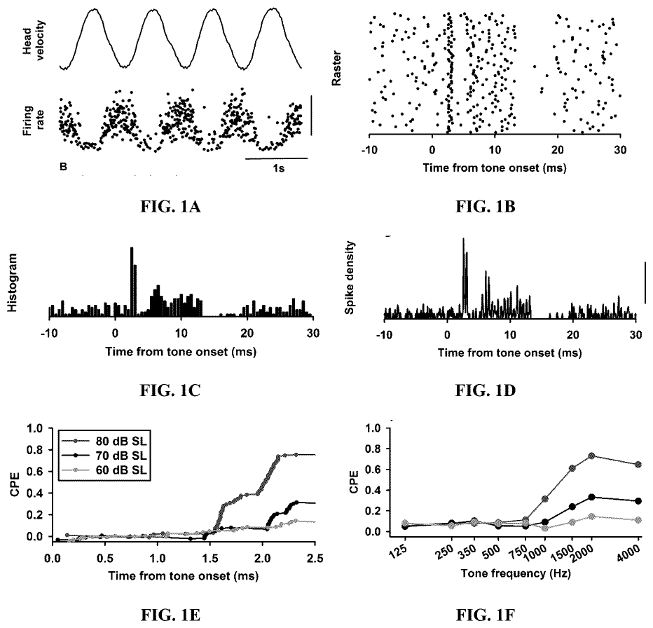

The cumulative probability of evoking a spike (CPE) was calculated as a quantitative measure of vestibular afferent sound sensitivity. Protocols were adapted from Broussard and Lisberger (1992) and described in Zhu et al. (2011, 2014). Briefly, latencies of the first action potential were measured for 30 ms before and 50 ms after onset of a tone-burst. The stimulus was repeated 150 times and the latencies were arranged in ascending order and paired with a number indicating probability of firing ranging from 0.007 to 1.0 in equal increments. Probability of firing was plotted as a function of the latency (sound condition). The spontaneous firing effect was assessed by making the same plot for 30 ms before the tone onset (no-sound condition). The CPE was calculated by subtracting the Y-value of the linear regression line of the no-sound condition from the sound condition (FIG. 1E). The response latency was measured as the onset of the abrupt increase in CPE; the response amplitude was measured as the height of the rapid change in CPE (difference between the peak probability and the baseline probability). An afferent was classified as a sound sensitive unit if its CPE was greater than 0.1 for at least one tone burst condition.

### Statistical Analysis

Statistical significance was assessed using the t-test or ANOVA (SigmaPlot 12.3 or Matlab R2020a). Multiple-comparison corrections (e.g. Student-Newman-Keuls test) were performed when needed. Statistical significance was determined based on P values of less than 0.05. Error bars were standard errors of the mean (SEM).

### Modeling Methods

A geometrical model of the rat bony labyrinth was constructed by segmenting micro CT data. Fast acoustic wave propagation and fluid motion inside the bony labyrinth was estimated computationally using the finite element method (COMSOL Multiphysics, Burlington, MA; Iversen, 2021). In the simulations, the bony labyrinth was filled with fluid and driven by an applied sinusoidal pressure at the stapes (125-4000 Hz), with pressure relief occurring at the compliant round window. Bone was assumed rigid, and the physical properties of the inner ear fluids (density, viscosity, bulk modulus) were approximated as equal to water at 36.5° C. (Steer et al., 1967; Money et al., 1971). The cochlear partition and the membranous labyrinth were not explicitly modeled, and therefore the model only addressed bulk fluid motion between the oval and round windows plus acoustic wave propagation in the fluids, without considering the potential contribution of traveling waves on the membrane labyrinth. The round window mechanical impedance was set to 2000-10⁷j/f(kg-m⁻²-s⁻¹) over a radius of 250 μm, where j=√(−1) and f is frequency (Hz). The experimentally observed peak sensitivity of ˜1500 Hz is sensitive to the round window stiffness, which is the imaginary part of the impedance. The average fluid velocity inside each semicircular canal ampulla, above the crista was computed as an indicator of the stimulus activating the canals, and the average fluid velocity within the bony labyrinth in volumes surrounding the utricular macula and the saccular macula was computed as an indicator of the stimulus activating the otolith organs (Iversen, 2021).

### Results

This report is based on tone-evoked responses of 1390 vestibular afferents from 99 Sprague-Dawley rats, including 342 anterior canal (AC), 238 horizontal canal (HC), 311 posterior canal (PC), 299 superior branch otolith (SO, primarily utricular) and 200 inferior branch otolith (IO, saccular) afferent neurons.

FIG. 1A shows the response of a representative irregular HC afferent neuron to sinusoidal head rotation at 1 Hz. This unit had a spontaneous firing rate of 109.2 spike/s and a CV* of 0.86. The gain (sensitivity) to 1 Hz sinusoidal head rotation was 1.27 spike/s per deg/s with a phase lead of 22.7 deg. Responses of the same HC afferent to a series of 2000 Hz tone bursts are shown in FIG. 1B as spike times in raster form, in FIG. 1C as a stimulus triggered histogram, and in FIG. 1D as spike density function (FIG. 1D, by convolving the spike train with a Gaussian with SD of 0.1 ms, Cullen et al., 1996) and CPE (FIG. 1F). 2000 Hz tone bursts evoked an increased discharge rate during the tone (FIGS. 1B-D) followed by a period of decreased firing rate. Examination of the spike density function revealed two additional features of the afferent response to 2000 Hz tones (FIG. 1D). First, there were multiple peaks in the response, which were separated by ˜0.5 ms corresponding to the period of the 2000 Hz tone and demonstrating synchronization of firing to the stimulus frequency. Second, the initial peak (805.2 spike/s at a latency of ˜2.4 ms) was much larger than the subsequent peak (227.9 spike/s at a latency of ˜2.9 ms). The time-dependent CPE for this afferent revealed an onset latency of 1.5 ms (FIG. 1E) reaching a stimulus-level-dependent peak at a latency of ˜2.4 ms after the tone onset. For the PC crista, the onset latency was considerably longer than synaptic delay (Rabbitt et al., 2016, 1995), revealing presence of a propagation delay between the sound stimulus trigger and hair bundle displacement in the HC crista. Peak CPE was used to quantify sensitivity to tone-bursts delivered at differing frequencies (125-4000 Hz) and intensities (60, 70 and 80 dB SL re ABR threshold), shown in FIG. 1F for this specific HC afferent. The HC afferent in FIG. 1F did not respond to tones lower than 750 Hz (≤80 dB SL), and responded with CPE<0.2 at all frequencies tested at 60 dB SL.

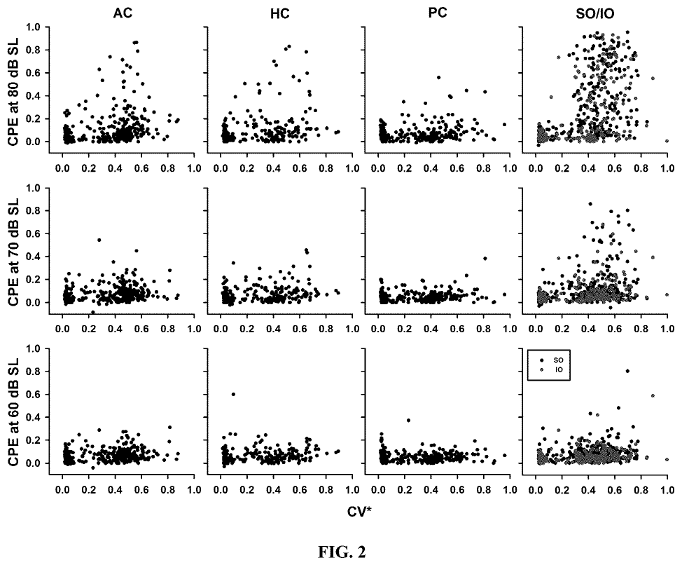

Afferents with irregular inter-spike intervals at rest were much more likely to respond to tone bursts than afferents with regular inter-spike intervals. FIG. 2 reports sensitivities of all afferents tested to 350 Hz tone bursts in the form of CPE vs. CV*. Results are shown for afferents innervating each of the canals (AC, HC, and PC) and for afferents innervating the otolith organs (SO, black symbols; IO, red symbols). Dots represent individual afferents, with dots higher on the vertical axis corresponding to a stronger response to the acoustic stimulus. Responses increased as the sound intensity was increased from 60 dB SL to 80 dB SL, with the otolith organs responding to a 350 Hz 70 dB SL tone burst with average CPE similar to the AC and HC at 80 dB SL. PC responses at 80 dB SL were similar to otolith responses at 60 dB SL. Results suggest that the otolith organs can be preferentially activated by 350 Hz tone bursts without activating the canals in the rat model by using stimuli ≤70 dB SL.

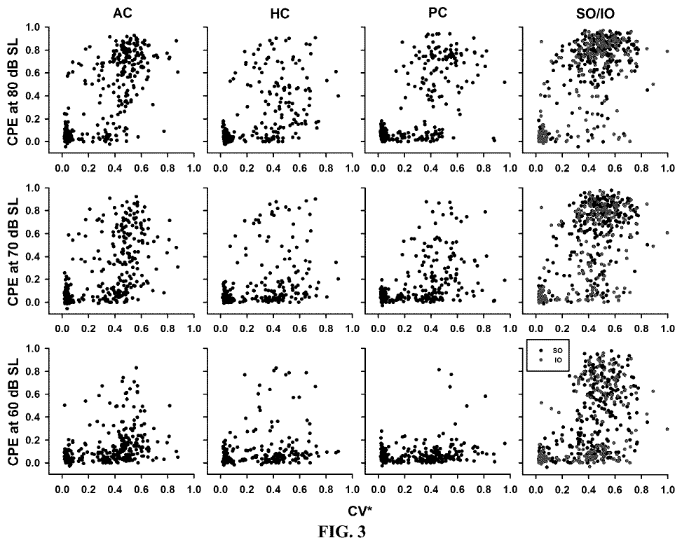

Both canal and otolith afferent neurons became more sensitive as the frequency was increased to 1500 Hz, with scatter plots of CPE vs CV* provided in FIG. 3 for the same units shown in FIG. 2. Similar to results at 350 Hz, the canals required a tone burst stimulus intensity 10-20 dB higher than the otoliths to evoke comparable CPE, once again suggesting the otolith organs can be preferentially activated over the canals by titrating the intensity of the stimulus to avoid activation of the canals. The precise level depends on frequency, and is likely to vary across species. It is important to note that CPE saturates at 1, suggesting CPE likely approaches 1 for sensitive irregularly discharging afferents in both the canals and otolith organs if the sound intensity is sufficiently high.

### Determinants of Afferent Sound Sensitivity

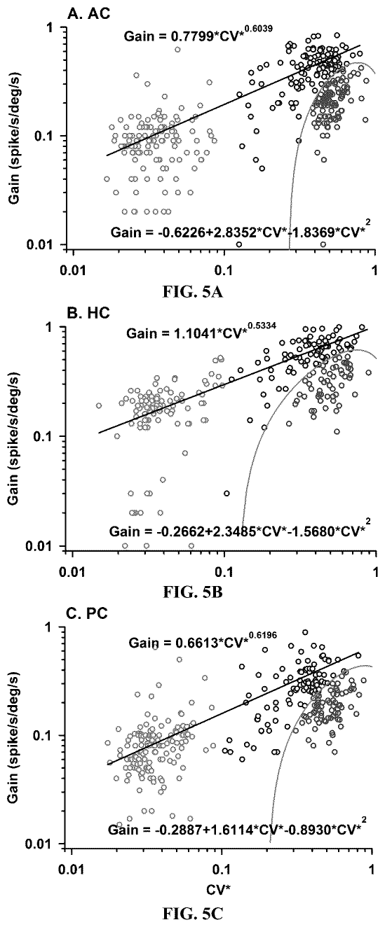

Based on the large data set, how properties of an afferent predict its sound sensitivity, i.e., regularity of spontaneous firing (CV*), origin (end organ), spontaneous firing rate and, in canal afferents sensitivity to head rotation were assessed. Average spontaneous firing rate, CV*, gain to head rotation of sound sensitive (SS) afferents, and non-sound sensitive (NSS) afferents were calculated separately for regular and irregular afferents of different vestibular end organs (Table 1). Irregular afferents were further divided into putative calyx afferents and non-calyx (dimorphic and bouton) afferents based on head rotation gain and regularity (FIGS. 5A-C and Table 2) (Baird et al., 1988; Marlinski et al., 2004). Multiple linear regressions were performed using CPE as the dependent variable and spontaneous firing rate, CV* and head rotation gain as independent variables to determine which parameters are predictors of afferent sound sensitivity.

TABLE 1
Sound sensitivities of the regular and irregular afferents of different vestibular end organs.

| End organ | | Sound | Firing Rate | Gain | | CPE 350 Hz |
|---|---|---|---|---|---|---|
| (N) | | Sensitivity | (spike/s) | (spike/s/deg/s) | CV* | 80 dB SL |
| AC | Regular | SS (14) | 132.16 ± 26.40 | 0.165 ± 0.040 | 0.052 ± 0.007 | 0.069 ± 0.027 |
| (342) | (120) | NSS (106) | 76.00 ± 1.98 | 0.093 ± 0.005 | 0.036 ± 0.001 | −0.024 ± 0.003 |
| | | Mean ± SE | 82.55 ± 3.83 | 0.101 ± 0.006 | 0.037 ± 0.001 | −0.013 ± 0.005 |
| | Irregular | SS (184) | 119.25 ± 4.30 | 0.330 ± 0.013 | 0.485 ± 0.010 | 0.098 ± 0.012 |
| | (222) | NSS (38) | 49.04 ± 4.26 | 0.336 ± 0.030 | 0.337 ± 0.031 | −0.048 ± 0.003 |
| | | Mean ± SE | 107.23 ± 4.04 | 0.331 ± 0.012 | 0.460 ± 0.011 | 0.073 ± 0.011 |
| | Mean ± SE | | 98.57 ± 3.01 | 0.250 ± 0.010 | 0.312 ± 0.013 | 0.042 ± 0.007 |
| HC | Regular | SS(13) | 157.53 ± 32.87 | 0.220 ± 0.021 | 0.051 ± 0.010 | 0.082 ± 0.027 |
| (238) | (97) | NSS (84) | 72.95 ± 1.81 | 0.185 ± 0.011 | 0.040 ± 0.001 | −0.025 ± 0.003 |
| | | Mean ± SE | 84.28 ± 5.40 | 0.190 ± 0.010 | 0.041 ± 0.002 | −0.010 ± 0.005 |
| | Irregular | SS(106) | 126.59 ± 5.95 | 0.466 ± 0.022 | 0.484 ± 0.017 | 0.107 ± 0.018 |
| | (141) | NSS(35) | 51.53 ± 4.02 | 0.541 ± 0.042 | 0.324 ± 0.025 | −0.046 ± 0.003 |
| | | Mean ± SE | 107.96 ± 5.33 | 0.484 ± 0.020 | 0.444 ± 0.015 | 0.069 ± 0.014 |
| | Mean ± SE | | 98.31 ± 3.92 | 0.364 ± 0.015 | 0.280 ± 0.015 | 0.036 ± 0.009 |
| PC | Regular | SS(2) | 80.15 ± 9.85 | 0.075 ± 0.005 | 0.029 ± 0.006 | 0.046 ± 0.003 |
| (311) | (142) | NSS(140) | 73.13 ± 1.46 | 0.090 ± 0.006 | 0.037 ± 0.001 | −0.012 ± 0.003 |
| | | Mean ± SE | 73.23 ± 1.45 | 0.090 ± 0.006 | 0.037 ± 0.001 | −0.012 ± 0.003 |
| | Irregular | SS(112) | 103.75 ± 5.20 | 0.282 ± 0.018 | 0.481 ± 0.015 | 0.034 ± 0.009 |
| | (169) | NSS(57) | 58.02 ± 4.80 | 0.266 ± 0.024 | 0.334 ± 0.021 | −0.034 ± 0.005 |
| | | Mean ± SE | 88.32 ± 4.15 | 0.277 ± 0.014 | 0.431 ± 0.013 | 0.011 ± 0.007 |
| | Mean ± SE | | 81.43 ± 2.38 | 0.191 ± 0.010 | 0.251 ± 0.013 | 0.001 ± 0.004 |
| SO | Regular | SS(6) | 147.41 ± 42.54 | 0.036 ± 0.020 | 0.059 ± 0.012 | 0.024 ± 0.030 |
| (299) | (48) | NSS(42) | 66.64 ± 3.18 | 0.015 ± 0.003 | 0.037 ± 0.002 | −0.026 ± 0.005 |
| | | Mean ± SE | 76.74 ± 6.85 | 0.017 ± 0.003 | 0.040 ± 0.002 | −0.020 ± 0.006 |
| | Irregular | SS(239) | 116.93 ± 4.02 | 0.079 ± 0.003 | 0.505 ± 0.013 | 0.307 ± 0.018 |
| | (251) | NSS(12) | 67.51 ± 9.69 | 0.047 ± 0.011 | 0.355 ± 0.096 | −0.040 ± 0.004 |
| | | Mean ± SE | 114.57 ± 3.91 | 0.077 ± 0.003 | 0.498 ± 0.013 | 0.290 ± 0.018 |
| | Mean ± SE | | 108.49 ± 3.55 | 0.068 ± 0.003 | 0.425 ± 0.015 | 0.240 ± 0.016 |
| IO | Regular | SS(5) | 81.05 ± 11.64 | 0.019 ± 0.008 | 0.043 ± 0.005 | −0.005 ± 0.009 |
| (200) | (49) | NSS(44) | 59.95 ± 3.10 | 0.011 ± 0.001 | 0.044 ± 0.002 | −0.031 ± 0.005 |
| | | Mean ± SE | 62.11 ± 3.12 | 0.012 ± 0.002 | 0.044 ± 0.002 | −0.028 ± 0.004 |
| | Irregular | SS(140) | 110.24 ± 5.74 | 0.088 ± 0.007 | 0.517 ± 0.017 | 0.289 ± 0.023 |
| | (151) | NSS(11) | 73.84 ± 15.89 | 0.080 ± 0.015 | 0.397 ± 0.067 | −0.033 ± 0.006 |
| | | Mean ± SE | 107.59 ± 5.49 | 0.087 ± 0.006 | 0.508 ± 0.016 | 0.265 ± 0.023 |

| | | | | |
|---|---|---|---|---|
| Mean ± SE | 96.45 ± 4.43 | 0.069 ± 0.005 | 0.394 ± 0.019 | 0.193 ± 0.016 |

| End organ | | Sound | CPE 1500 Hz | CPE 350 Hz | CPE 1500 Hz |
|---|---|---|---|---|---|
| (N) | | Sensitivity | 80 dB SL | 60 dB SL | 60 dB SL |
| AC | Regular | SS (14) | 0.287 ± 0.066 | 0.019 ± 0.015 | 0.072 ± 0.031 |
| (342) | (120) | NSS (106) | −0.0212 ± 0.003 | −0.022 ± 0.003 | −0.024 ± 0.003 |
| | | Mean ± SE | 0.0148 ± 0.012 | −0.017 ± 0.003 | −0.012 ± 0.005 |
| | Irregular | SS (184) | 0.5699 ± 0.015 | 0.014 ± 0.004 | 0.124 ± 0.013 |
| | (222) | NSS (38) | −0.0268 ± 0.007 | −0.045 ± 0.004 | −0.042 ± 0.003 |
| | | Mean ± SE | 0.4678 ± 0.019 | 0.003 ± 0.004 | 0.095 ± 0.011 |
| | Mean ± SE | | 0.3089 ± 0.017 | −0.003 ± 0.003 | 0.057 ± 0.008 |
| HC | Regular | SS(13) | 0.144 ± 0.057 | 0.073 ± 0.043 | 0.044 ± 0.022 |
| (238) | (97) | NSS (84) | −0.023 ± 0.003 | −0.026 ± 0.003 | −0.027 ± 0.004 |
| | | Mean ± SE | −0.001 ± 0.009 | −0.013 ± 0.007 | −0.017 ± 0.005 |
| | Irregular | SS(106) | 0.381 ± 0.024 | 0.009 ± 0.005 | 0.118 ± 0.021 |
| | (141) | NSS(35) | −0.031 ± 0.006 | −0.038 ± 0.005 | −0.044 ± 0.005 |
| | | Mean ± SE | 0.279 ± 0.023 | −0.002 ± 0.004 | 0.078 ± 0.017 |
| | Mean ± SE | | 0.164 ± 0.017 | −0.006 ± 0.004 | 0.039 ± 0.010 |
| PC | Regular | SS(2) | 0.039 ± 0.028 | −0.005 ± 0.025 | 0.041 ± 0.040 |
| (311) | (142) | NSS(140) | −0.012 ± 0.002 | −0.016 ± 0.003 | −0.011 ± 0.003 |
| | | Mean ± SE | −0.011 ± 0.002 | −0.016 ± 0.003 | −0.010 ± 0.003 |
| | Irregular | SS(112) | 0.573 ± 0.017 | −0.006 ± 0.004 | 0.039 ± 0.012 |
| | (169) | NSS(57) | −0.009 ± 0.014 | −0.040 ± 0.003 | −0.029 ± 0.006 |
| | | Mean ± SE | 0.376 ± 0.024 | −0.017 ± 0.003 | 0.016 ± 0.009 |
| | Mean ± SE | | 0.199 ± 0.017 | −0.017 ± 0.002 | 0.004 ± 0.005 |
| SO | Regular | SS(6) | 0.231 ± 0.074 | 0.073 ± 0.038 | 0.001 ± 0.055 |
| (299) | (48) | NSS(42) | −0.029 ± 0.004 | −0.027 ± 0.005 | −0.025 ± 0.006 |
| | | Mean ± SE | 0.003 ± 0.015 | −0.015 ± 0.008 | −0.022 ± 0.008 |
| | Irregular | SS(239) | 0.677 ± 0.011 | 0.022 ± 0.005 | 0.389 ± 0.018 |
| | (251) | NSS(12) | −0.033 ± 0.009 | −0.039 ± 0.006 | −0.055 ± 0.007 |
| | | Mean ± SE | 0.643 ± 0.014 | 0.019 ± 0.005 | 0.368 ± 0.018 |
| | Mean ± SE | | 0.540 ± 0.018 | 0.014 ± 0.004 | 0.305 ± 0.017 |
| IO | Regular | SS(5) | 0.384 ± 0.116 | −0.032 ± 0.001 | 0.069 ± 0.096 |
| (200) | (49) | NSS(44) | −0.034 ± 0.005 | −0.028 ± 0.004 | −0.031 ± 0.005 |
| | | Mean ± SE | 0.008 ± 0.021 | −0.029 ± 0.004 | −0.021 ± 0.010 |
| | Irregular | SS(140) | 0.662 ± 0.017 | 0.006 ± 0.006 | 0.362 ± 0.027 |
| | (151) | NSS(11) | −0.039 ± 0.007 | −0.032 ± 0.009 | −0.033 ± 0.009 |
| | | Mean ± SE | 0.611 ± 0.021 | 0.003 ± 0.006 | 0.333 ± 0.026 |

| | | | |
|---|---|---|---|
| Mean ± SE | 0.463 ± 0.025 | −0.004 ± 0.004 | 0.246 ± 0.022 |

TABLE 2
Sound sensitivities of the putative calyx and dimorphic afferents.

| Vestibular | | Sound | Firing Rate | Gain | | CPE 350 Hz | CPE 1500 Hz | CPE 350 Hz | CPE 1500 Hz |
|---|---|---|---|---|---|---|---|---|---|
| afferent (N) | | sensitivity | (spike/s) | (spike/s/deg/s) | CV* | 80 dB SL | 80 dB SL | 60 dB SL | 60 dB SL |
| AC | Dimorphic | SS(87) | 117.01 ± 7.54 | 0.467 ± 0.018 | 0.424 ± 0.014 | 0.079 ± 0.019 | 0.518 ± 0.024 | 0.009 ± 0.005 | 0.101 ± 0.018 |
| (220) | (114) | NSS(27) | 53.99 ± 5.31 | 0.401 ± 0.033 | 0.306 ± 0.018 | 0.047 ± 0.005 | 0.026 ± 0.010 | 0.041 ± 0.005 | 0.042 ± 0.004 |
| | | Mean ± SE | 102.09 ± 6.39 | 0.452 ± 0.016 | 0.396 ± 0.012 | 0.049 ± 0.015 | 0.389 ± 0.028 | 0.002 ± 0.004 | 0.067 ± 0.015 |
| | Calyx | SS(98) | 119.54 ± 4.66 | 0.207 ± 0.007 | 0.535 ± 0.014 | 0.117 ± 0.016 | 0.615 ± 0.016 | 0.019 ± 0.006 | 0.143 ± 0.017 |
| | (106) | NSS(8) | 63.64 ± 15.33 | 0.211 ± 0.038 | 0.542 ± 0.101 | 0.052 ± 0.005 | 0.020 ± 0.010 | 0.054 ± 0.005 | 0.041 ± 0.007 |
| | | Mean ± SE | 115.32 ± 4.67 | 0.208 ± 0.007 | 0.536 ± 0.015 | 0.105 ± 0.015 | 0.567 ± 0.022 | 0.014 ± 0.006 | 0.129 ± 0.017 |
| HC | Dimorphic | SS(53) | 128.48 ± 10.46 | 0.627 ± 0.027 | 0.457 ± 0.024 | 0.075 ± 0.019 | 0.314 ± 0.032 | 0.012 ± 0.007 | 0.070 ± 0.024 |
| (139) | (77) | NSS(24) | 52.31 ± 5.45 | 0.676 ± 0.032 | 0.357 ± 0.019 | 0.050 ± 0.003 | 0.038 ± 0.006 | 0.041 ± 0.004 | 0.046 ± 0.004 |
| | | Mean ± SE | 104.74 ± 8.41 | 0.643 ± 0.021 | 0.426 ± 0.018 | 0.036 ± 0.014 | 0.204 ± 0.029 | 0.004 ± 0.006 | 0.033 ± 0.018 |
| | Calyx | SS(55) | 124.47 ± 7.15 | 0.300 ± 0.015 | 0.497 ± 0.024 | 0.141 ± 0.029 | 0.445 ± 0.032 | 0.009 ± 0.006 | 0.164 ± 0.032 |
| | (62) | NSS(7) | 65.10 ± 19.73 | 0.240 ± 0.045 | 0.350 ± 0.083 | 0.040 ± 0.013 | 0.002 ± 0.022 | 0.031 ± 0.019 | 0.040 ± 0.020 |
| | | Mean ± SE | 117.76 ± 7.09 | 0.293 ± 0.014 | 0.481 ± 0.024 | 0.120 ± 0.027 | 0.395 ± 0.034 | 0.005 ± 0.006 | 0.141 ± 0.029 |
| PC | Dimorphic | SS(50) | 109.16 ± 10.49 | 0.391 ± 0.034 | 0.387 ± 0.021 | 0.022 ± 0.013 | 0.535 ± 0.028 | 0.001 ± 0.008 | 0.015 ± 0.012 |
| (169) | (99) | NSS(49) | 54.31 ± 4.73 | 0.260 ± 0.018 | 0.300 ± 0.017 | 0.035 ± 0.005 | 0.024 ± 0.005 | 0.040 ± 0.003 | 0.031 ± 0.006 |
| | | Mean ± SE | 82.01 ± 6.39 | 0.324 ± 0.020 | 0.344 ± 0.014 | 0.006 ± 0.007 | 0.258 ± 0.031 | 0.021 ± 0.004 | 0.007 ± 0.007 |
| | Calyx | SS(63) | 99.65 ± 5.20 | 0.192 ± 0.009 | 0.552 ± 0.016 | 0.043 ± 0.013 | 0.594 ± 0.024 | 0.010 ± 0.004 | 0.057 ± 0.020 |
| | (70) | NSS(7) | 86.42 ± 16.67 | 0.337 ± 0.152 | 0.577 ± 0.081 | 0.034 ± 0.006 | 0.090 ± 0.110 | 0.037 ± 0.011 | 0.013 ± 0.025 |
| | | Mean ± SE | 98.33 ± 4.95 | 0.208 ± 0.017 | 0.555 ± 0.016 | 0.036 ± 0.012 | 0.544 ± 0.030 | 0.013 ± 0.004 | 0.050 ± 0.018 |

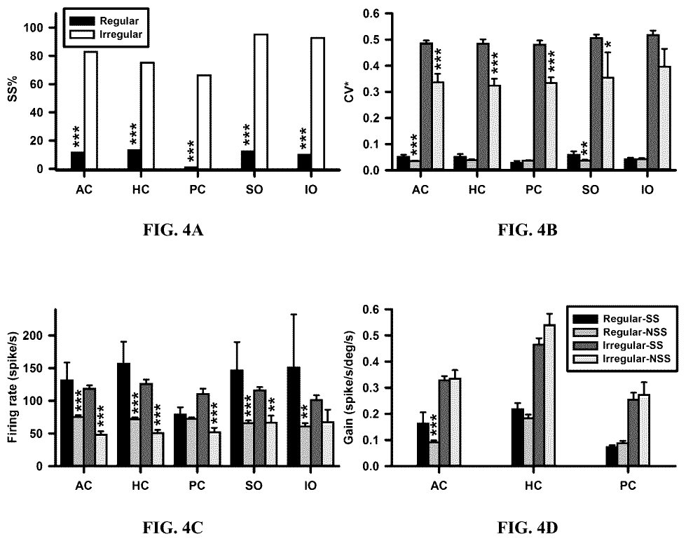

Regularity of spontaneous firing. Vestibular afferents with irregular inter-spike intervals (CV*≥0.1) were much more likely to respond to tone bursts with a high CPE (FIGS. 3 and 4A-B). To demonstrate this correlation, individual afferents were categorized as sound sensitive (SS, CPE≥0.1) or not sound sensitive (NSS, CPE<0.1). FIG. 4A summarizes the percentages of SS regular and SS irregular afferents for each vestibular end organ at 80 dB SL. Overall, 8.8% (40/456) of regularly discharging afferents were sound sensitive, while 83.6% (781/934) of irregularly discharging afferents were sound sensitive. For regularly discharging afferents, the percentages exhibiting sound sensitivity was 11.7%, 13.4%, 1.4%, 12.5% and 10.2% for AC, HC, PC, SO and IO, respectively. Note that PC had the lowest percentage of sound sensitive regular afferents compared to the other end organs. For irregularly discharging afferents, the percentages exhibiting sound sensitivity was 82.9%, 75.2%, 66.3%, 95.2% and 92.7% for AC, HC, PC, SO and IO, respectively. Differences between organs are likely to reflect differences in the mechanical stimulus deflecting hair bundles (Curthoys et al., 2019, 2016) in addition to differences in frequency response and encoding properties. On average across all organs, irregularly discharging afferents were nearly 10 times more likely to be activated by tone bursts relative to their regularly discharging counterparts.

Vestibular end organ. The second important factor that determines sound sensitivity of a vestibular afferent is its end organ. Summary data in FIG. 4A shows that irregularly discharging otolith afferents (SO/IO) had higher percentages of SS afferents than irregularly discharging canal afferents (AC/HC/PC). Overall, 48.4% (431/891) of canal afferents were SS, while 78.2% (390/499) of otolith afferents were SS (Chi-square=116.125 with 1 degrees of freedom, P<0.001) at 80 dB SL across the frequency bandwidth tested.

Spontaneous firing rate and gain to rotation. In addition to CV* and origin of end organ, irregularly discharging afferent neurons with high spontaneous discharge rates were more likely to be sound sensitive than irregularly discharging afferents with lower spontaneous discharge rates. FIG. 4C shows that spontaneous firing rates of SS afferents were about twice of that of NSS afferents. For example, in the irregular afferents, the averaged baseline firing rates of SS afferents were 119.3±4.3, 126.6±6.0, 103.8±5.2, 116.9±4.0, and 110.2±5.7 spike/s for AC, HC, PC, SO and IO, respectively, whereas the averaged baseline firing rates of NSS afferents were 49.0±4.3, 51.5±4.0, 58.0±4.8, 67.5±9.9, and 73.8±15.8 spike/s for AC, HC, PC, SO and IO respectively. FIG. 4D shows that gains to 2 Hz head rotation were similar in the SS afferents and the NSS afferents.

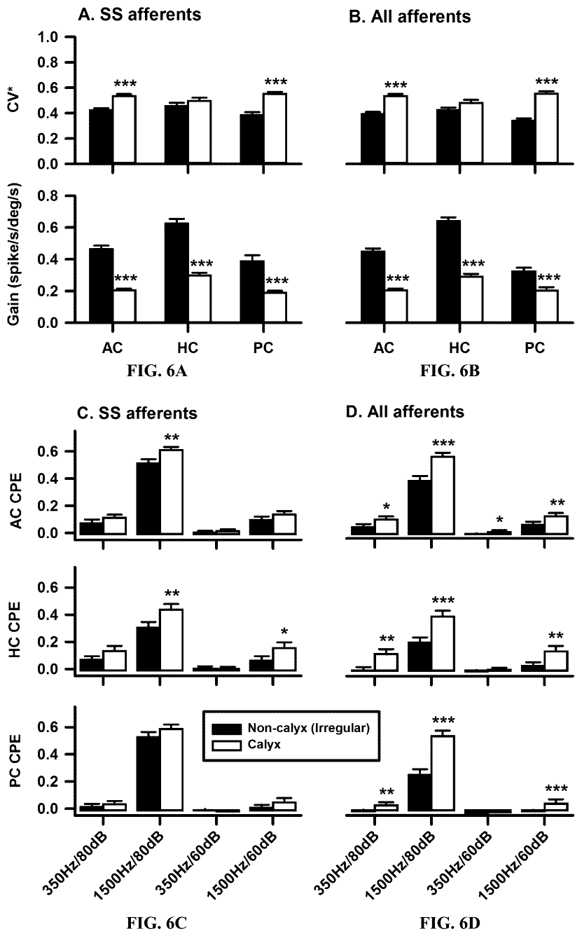

Terminal type as a predictor for sound sensitivity. The vestibular sensory epitheliums are located on the maculae of the saccule/utricle and the cristae of the semicircular canals and have two types of hair cells (Baird et al. 1988). Whereas Type I hair cells are in the central regions and are innervated by afferents with calyx terminals, Type II hair cells are in the peripheral regions and are innervated by afferents with bouton terminals. Based on the type of terminals, afferents are classified into three categories, bouton afferents, which only innervate Type II hair cells with bouton terminals, calyx afferents, which only innervate Type I hair cells with calyx terminals, and dimorphic afferents, which innervate both Type I and Type II hair cells with bouton and calyx terminals, respectively. Baird et al. (1988) and Marlinski et al. (2004) showed that the calyx afferents can be putatively identified in the plot of horizontal rotation gain (2 Hz) vs. CV*. FIGS. 5A-C show that the AC/HC/PC afferents were classified into the calyx units (red symbols) and non-calyx units (bouton and dimorphic units, blue and gray symbols) using the methods of Baird et al. (1988) and Marlinski et al. (2004). The properties of the putative calyx units and non-calyx units were then compared in the canals (FIGS. 6A-D and Table 2). The putative calyx units, which had higher CV* and lower rotational gains than the non-calyx units (FIGS. 5A-C and 6A-B), exhibited larger tone-evoked responses (i.e., CPE) than the non-calyx units (FIGS. 6C-D). The calyx canal afferents were more likely to be sound sensitive than that of the non-calyx canal afferents (90.8% vs 65.5%, Table 2). The higher CPEs of the putative calyx units were unlikely due to their higher irregularity because the averaged CV* of the NSS putative calyx units was similar to that of the SS calyx units in AC and PC afferents (CV*ss. vs. CV*NSS, AC: 0.534±0.104 vs. 0.542±0.101; PC: 0.552±0.016 vs. 0.577±0.081).

Multiple linear regression analysis. Regression analysis was performed for irregular canal afferents (AC/HC/PC) and irregular otolith afferents (SO/IO). For irregular canal afferents, the regression equation is:

$$CPE = 0.13 + 0.00161 \times FR + 0.646 \times {CV}^{*} - 0.343 \times G ( N = 532 , R = 0.539 , P < 0.001 )$$

Where CPE is tone burst evoked response (1500 Hz at 80 dB SL); FR is spontaneous firing rate; CV* is regularity of spontaneous firing and G is gain to head rotation at 2 Hz. Coefficients for FR, CV* and G have P-values <0.001. Although coefficient for head rotation gain was significant and negative, it should not be interpreted as predicting factor for CPE because it was resulted from the fact that the calyx units had lower head rotation gain, but larger CPE (FIG. 6 and Table 2). Within the putative calyx units and the noncalyx units, head rotation gain cannot account for the differences in CPE between SS and NSS afferents. For example, in putative AC calyx afferents, SS units had a gain of 0.207±0.007 spike/s/deg/s, but NSS units had a gain of 0.211±0.038 spike/s/deg/s. Regression analyses were further performed for the non-calyx canal afferents to confirm that head rotation gain was not a reliable factor to predict afferent sound sensitivity.

For AC non-calyx afferents, the regression equation is:

$$CPE = - 0.0901 + 0.00187 \times FR + 0.801 \times {CV}^{*} + 0.0934 \times G$$

(N=120, R=0.601, P<0.001; P-value of coefficients for FR and CV*<0.001, P-value of coefficient for G is 0.486).

For HC non-calyx afferents, the regression equation is:

$$CPE = 0.0629 + 0.00187 \times FR + 0.459 \times {CV}^{*} - 0.276 \times G$$

(N=86, R=0.644, P<0.001; P-value for coefficients for FR and CV*<0.001, P-value of coefficient for G is 0.021).

For irregular PC non-calyx afferents, the regression equation is:

$$CPE = - 0.0397 + 0.00212 \times FR + 0.694 \times {CV}^{*} - 0.175 \times G$$

(N=97, R=0.516, P<0.001; P-value of coefficient for FR<0.001, P-value of coefficient for CV* is 0.003, P-value of coefficient for Gain is 0.277).

For otolith afferents, the regression equation is:

$$CPE = 0.0214 + 0.000346 \times FR + 0.0445 \times {CV}^{*}$$

(N=402, R=0.33, P<0.001; P-value of coefficient for FR<0.001, P-value of coefficient for CV* is 0.013).

### Differential Activation of Canal and Otolith Afferents by Tone Bursts

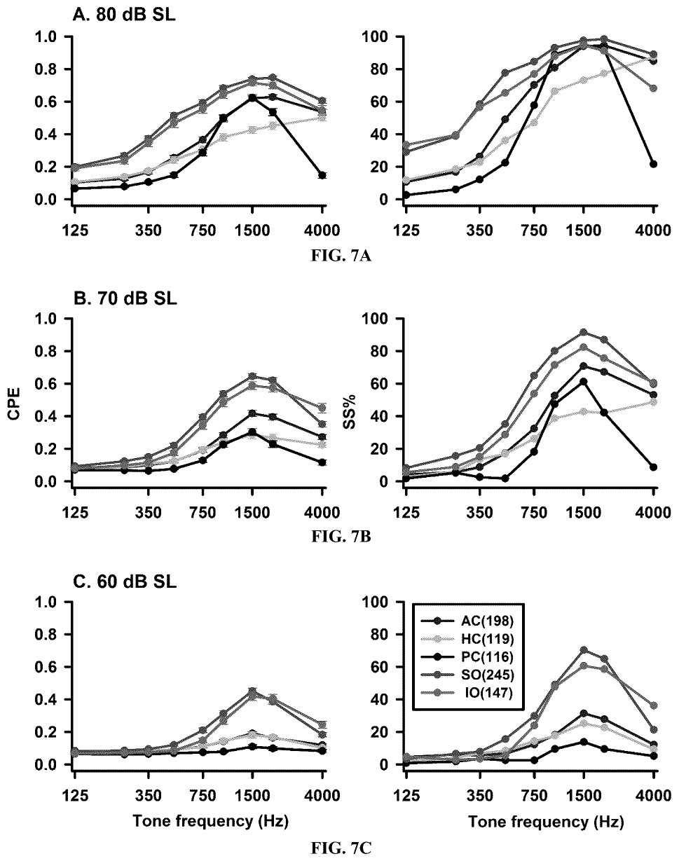

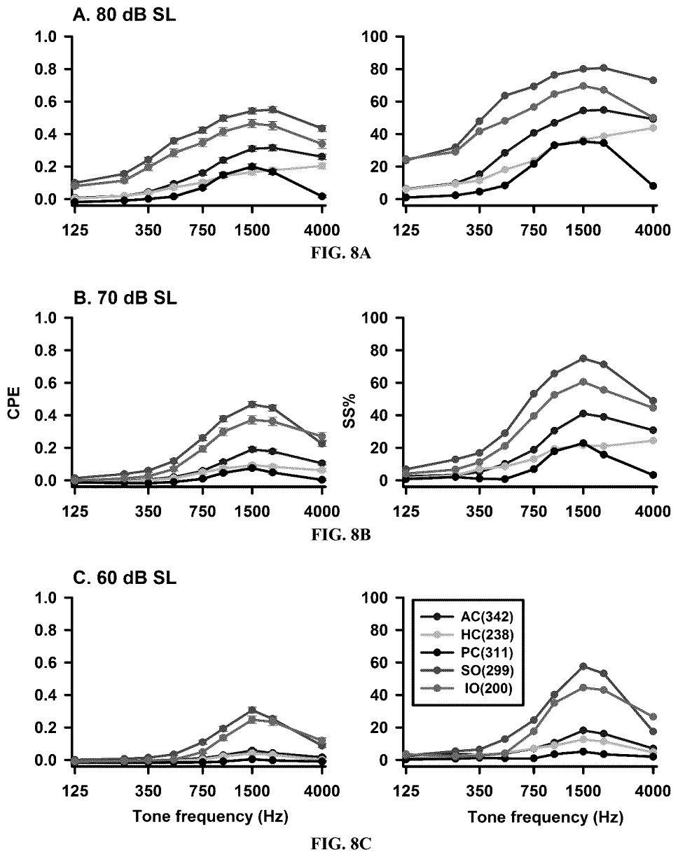

To quantitatively assess how tone bursts activate canal and otolith afferents, we calculated averaged CPEs of the SS afferents for each end organ and plotted them against tone frequency at each of the three intensity levels (FIGS. 7A-C left panels, error bars are within the symbols). The percentages of SS afferents were also calculated in each condition (FIGS. 7A-C right panels, error bars are within the symbols). The tuning curves were further calculated for the whole afferent population (SS and NSS afferents) from each end organ (FIGS. 8A-C, error bars are within the symbols). These tuning curves were well defined for each end organ and exhibited the following three basic features. First, between 125 Hz to 2000 Hz, sound sensitivity (i.e., CPE) increased with tone frequency. Between 2000 Hz to 4000 Hz, however, different end organs exhibited different responses: while sound sensitivity of the AC, SO and IO afferents reached peaks at 1500 Hz and slightly decreased at 4000 Hz, sound sensitivity of the HC afferents continued to increase with frequency; sound sensitivity of the PC afferents reached peaks at 1500 Hz and substantially decreased with frequency. In fact, at 4000 Hz, less than 10% of PC afferents were activated even at the highest intensity of 80 dB SL. Second, low frequency (<350 Hz) tone bursts did not activate canal afferents even at 80 dB SL, but can activate significant numbers of otolith afferents. For example, at 80 dB SL (FIG. 8A), tone bursts of 350 Hz activated about 40% of SO and IO afferents with averaged CPE of 0.217, but less than 10% of AC/HC/PC afferents with averaged CPE of 0.027. Third, tone bursts of 1500 Hz activated both canal and otolith afferents at high intensity, but they only activated the otolith afferents at low intensity. For example, at 80 dB SL (FIG. 8A), tone bursts of 1500 Hz activated about 75% otolith afferents with averaged population CPE of 0.502 and about 40% canal afferents with averaged population CPE of 0.224; at 60 dB SL (FIG. 8C), however, tone bursts of 1500 Hz activated about 50% otolith afferents with averaged population CPE of 0.276 and about 10% of canal afferents with averaged population CPE of 0.033.

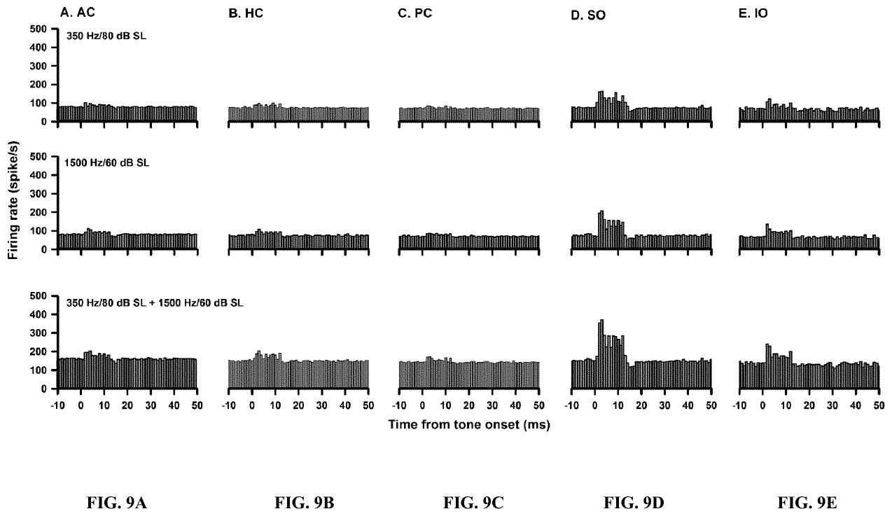

The differential activation of canal vs. otolith afferents is further illustrated in FIGS. 9A-E, which shows averaged post-tone burst onset histograms of canal afferents (FIGS. 9A-C) and otolith afferents (FIGS. 9D-E). Tone bursts of 350 Hz at 80 dB SL evoked an increase of 8.9 spike/s from a baseline of 76.7 spike/s in the population of canal afferents, while they evoked an increase of 35.8 spike/s from a baseline of 72.8 spike/s in the population of otolith afferents. Tone bursts of 1500 Hz at 60 dB SL evoked an increase of 13.2 spike/s from a baseline of 75.5 spike/s in the population of canal afferents, but an increase of 54.6 spike/s from a baseline of 72.0 spike/s in the population of otolith afferents. Assuming linear summation of tone-evoked responses in the afferents, combination of the two-tone bursts would evoke an increase of 11.05 spike/s (from a baseline of 76.1 spike/s) and an increase of 45.2 spike/s (from a baseline of 72.4 spike/s) in the population of canal and otolith afferents, respectively. In other words, about 80.4% of the two-tone bursts-evoked responses in the afferents are of otolith origin (FIGS. 9A-E).

### Latency of Tone Burst-Evoked Responses

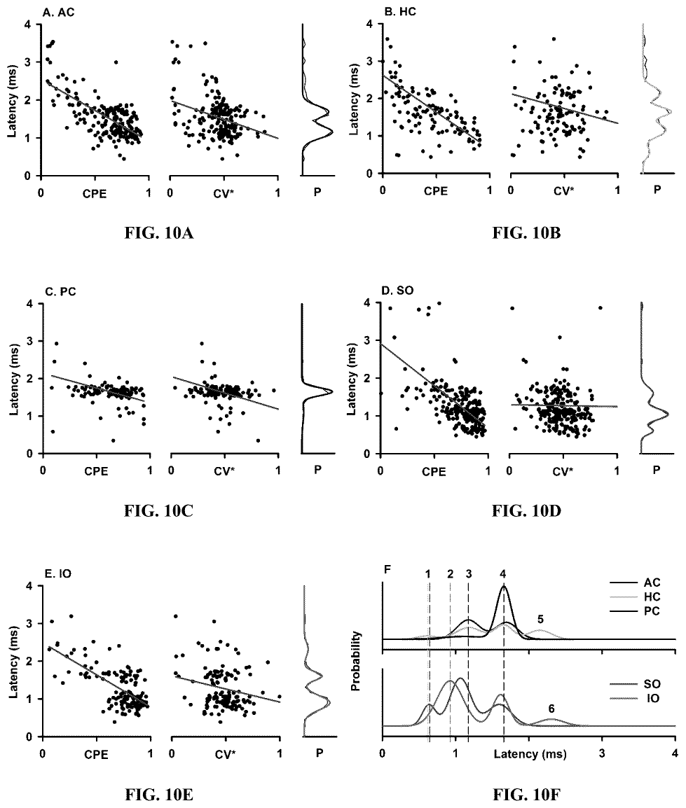

SS afferent neurons responded to tone bursts with latencies from ˜0.54 to 3 ms. The onset latencies of action potentials evoked by a 1500 Hz, 80 dB tone-burst are shown in FIGS. 10A-E for the AC, HC, PC, SO and IO. Individual data points show individual afferent neuron latency vs. peak CPE and spontaneous discharge regularity CV*. Neurons with high CPE responded to the tone at a specific phase during the stimulus cycle, resulting in horizontal bands in the scatter plots and multi-modal probability distributions (P) based on latency histograms of all units tested (P, solid curves on the right of each panel). The shortest latency band, or first peak in P, corresponds to an action potential evoked by the earliest stimulus cycle, while longer latency bands correspond to action potentials evoked after skipping one or more cycles. Latencies include the propagation delay as the sound reaches the organ “t₁”, plus the phase-locking delay “t₂” accounting for the specific time during the stimulus when the neuron fires. The total delay is t_{d}=t₁+k*t₂, where k is the number of cycles skipped between phase locked spikes (“k” is also called the winding ratio, WR Iversen et al. 2017, 2018). Shorter latencies in SO and HC neurons (FIG. 10F) suggest the propagation delay is shortest for sounds reaching these organs, while the long latency for PC afferents suggests a much longer propagation delay, as perhaps expected if excitation arises from a wave traveling along the membranous labyrinth to the PC ampulla (Iversen & Rabbitt, 2017). For the whole afferent population, latency distribution was clearly bimodal, with the first peak at 1.1 ms and the second peak at 1.7 ms (FIG. 10A). For afferents from individual end organs, bimodal distribution was observed for AC afferents (FIG. 10B, first peak at 1.1 ms, second peak at 1.7 ms), HC afferents (FIG. 10C, first peak at 1.1 ms, second peak at 1.6 ms), SO afferents (FIG. 10E, first peak at 1.1 ms, second peak at 1.7 ms) and IO afferents (FIG. 10F, first peak at 0.9 ms, second peak at 1.6 ms). However, PC afferents do not have a multimodal response pattern (FIG. 10D, one peak at 1.6 ms), but rather all seem to fire at once with a latency that is reliably greater than the AC and HC. More studies are needed to elucidate the mechanisms underlying the different latency distributions in PC and the other end organs.

The relationships between latency and CPE/CV* were examined by correlation analyses for SS afferents (FIGS. 10A-E). Latencies were significantly inversely correlated with CPEs for AC afferents (R=0.68, F (1,196)=170.0723, P<0.0001), HC afferents (R=0.55, F (1,117)=51.4717, P<0.0001), PC afferents (R=0.37, F (1,112)=18.1340, P<0.0001), SO afferents (R=0.62, F (1,243)=153.0712, P<0.0001) and IO afferents (R=0.67, F (1,143)=119.9582, P<0.0001) afferents, i.e., the larger the response, the shorter the latency. Latencies were also significantly inversely correlated with CV* for AC afferents (R=0.34, F (1,196)=26.4720, P<0.0001), HC afferents (R=0.19, F (1,117)=4.1884, P=0.0429), PC afferents (R=0.35, F (1,112)=15.6614, P=0.0001) and IO afferents (R=0.29, F (1,143)=12.9606, P=0.0004) afferents, i.e., the more irregular the spontaneous discharge, the shorter the latency. No significant correlation was found for the SO afferents (R=0.02, F (1,243)=0.0660, P=0.7974).

Although the average latency decreases with increasing CPE in some organs (e.g. AC, SO, HC), this relationship likely reflects a decrease winding ratio “k” with increasing CPE, not an actual change in the time when a spike is evoked relative to the phase of the sinusoidal stimulus. This is evidenced by organization of responses in horizontal bands, with each band largely independent of CPE and CV*. Considering that the central nervous system receives inputs from all of these afferent neurons at the same time, the distribution of spike times illustrated for the population in FIG. 10F is most relevant to tone-burst evoked vestibular reflexes.

### Finite Element (FE) Modeling of the Rat Inner Ear

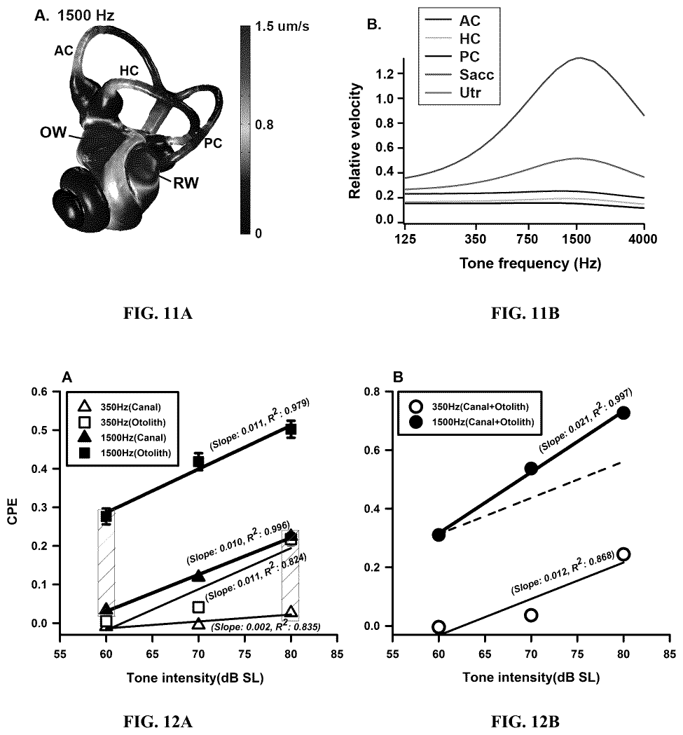

FIG. 11A shows the distribution of fluid velocity at the surface of the bony labyrinth when driving the oval window with sinusoidal at 1500 Hz (μm/s, black-red). Most of the fluid motion is predicted to be trapped in the vicinity of the oval and round windows as evanescent fluid motion, resulting in activation primarily of the utricle and saccule (FIG. 11A). To estimate activation of each organ, the fluid velocity was averaged over the anatomical location defining the surface of each macula and across the cross section of each ampulla. Normalized velocities exciting each organ predicted by this model are plotted in FIG. 11B as a function of stimulus frequency. Averaging across organs, frequency dependence predicted by this simple model is remarkably similar to experiments (FIGS. 7A-8C) showing a broadband tuning with a peak centered near 1500 Hz.

### DISCUSSION

Present results determine the relative sensitivities of identified afferent neurons innervating the semicircular canals and otolith organs to air-conducted tone-bursts in rats, over a frequency bandwidth from 125-4000 Hz and sound pressure range from 60-80 dB SL. On average, the cumulative probability of evoking a spike increased with increasing spontaneous discharge irregularity, increased with increasing sound level, and was broadly tuned with peak sensitivity near 1500 Hz. Results suggest differences in sensitivity and tuning arise from biophysical properties of the hair-cell-afferent complexes and biomechanical properties of vestibular organ activation by sound.

Direct evidence of the biophysical contribution to sound sensitivity is that afferent neurons with irregularly spaced inter-spike intervals are far more likely to be activated by tone bursts than neurons with regularly spaced interspike intervals (present data; Curthoys et al., 2011, 2019). Irregularly discharging afferent neurons in rats were nearly 10 times more likely to be sound sensitive than regularly discharging afferents (83.6% vs. 8.8%, FIG. 4A). The otolith afferent neurons were more likely to be activated by tone bursts than the canal afferent neurons (78.2% vs. 48.4%, FIG. 4A). Among the three canals, the irregular AC afferent neurons are the most sensitive to sound. In fact, the percentage of AC SS afferent neurons is similar to that of the irregular otolith afferent neurons (84% vs 94%). Spontaneous discharge rate is also correlates to afferent sound sensitivity. The average spontaneous firing rate of SS afferent neurons was about twice of that of NSS afferent neurons (SS: 115.3±8.7 spike/s; NSS: 60.0±10.5 spike/s; P<0.0001). Results support the hypothesis that in addition to micromechanical specializations within the organ of origin, sound sensitivity also depends on biophysical specializations associated with irregularly discharging afferents and associated synaptic inputs. It is well established that spontaneous discharge regularity in vestibular afferents correlates strongly with synapse type and channel properties (Lysakowski and Goldberg, 1997). Afferents exhibiting irregular inter-spike intervals make calyceal synaptic contacts on type I hair cells, while units with regular inter-spike intervals make bouton contacts on type II hair cells. Intracellular labeling of afferents in guinea pig and rat confirm that afferents most sensitive to auditory frequency sound and vibration are calyx bearing (Zhu et al., 2014; Curthoys et al, 2016). The vestibular calyx synapse is unique in supporting glutamatergic quantal transmission and two forms of nonquantal transmission (Contini et al., 2020), the fastest of which is a resistive coupling mechanism that immediately depolarizes the postsynaptic calyx synaptic terminal in synchrony with depolarization of the presynaptic hair cell. Ultrafast synaptic transmission combined with biophysical properties of the spike generator in irregularly discharging afferents is likely required for exquisite auditory frequency sensitivity in these specific vestibular afferent neurons.

Biomechanics is also important because it determines how sound causes mechanical vibration of the vestibular organs and displacement of vestibular hair cell bundles. The fact that sound sensitivity of the semicircular canals increases dramatically following fenestration of the bony labyrinth is direct evidence of the important role played by mechanics in auditory frequency sensitivity of vestibular afferent neurons (Tullio, 1929; Carey et al., 2004; Curthoys et al., 2019; Iversen et al., 2018). Finite element (FE) modeling of the rat inner ear was used to examine how air conducted sound might have activated the vestibular organs in the present experiments. Fluid compressibility and 3D bony morphology was included in the analysis, but the cochlear partition and membranous labyrinth were not. Hence, results only provide estimates of the gross fluid vibration at the locations of the vestibular organs. Frequency tuning in this model arises primarily from fluid motion between the compliant oval and round windows, and secondarily from compressible acoustic wave propagation. Simulations predict the saccule (FIG. 11B, red) receives the most mechanical excitation through stapes driven fluid motion. Excitation of the canals predicted by this model follows the same frequency dependence but is reduced in magnitude relative to the otolith organs. The fact that all organs are excited with similar frequency dependence is consistent with experimental data (FIGS. 7A-8C), but excitation of the canals in the simulations is lower than that measured experimentally. This difference is most likely due to the fact that the model did not include the membranous labyrinth and therefore missed vibration transferred from the otolith organs to the canals along the vibrating membranous labyrinth. Wave propagation along the vibrating membranous labyrinth is known to be important in dehiscence syndrome (Iversen et al., 2018; Iversen et al., 2017) and likely plays a role in the intact labyrinth as well. Precisely how local mechanics is related to afferent responses in otoliths vs. canals likely also contributes to the difference. Given simplifications in the model, the qualitative correspondence between the simulations (FIG. 11B) and data (FIGS. 7A-8C) suggests sound excites vestibular organs by simple mechanical vibration of each end organ and associated displacement of sensory hair bundles.

The FE model suggests that tone bursts excite the vestibular end organs through bursts of vibration deflecting hair bundles cycle-by-cycle. For a 2000 Hz stimulus, the period of each cycle is 0.5 ms, thus suggesting that stimulus-triggered histograms would reveal multiple peaks arising from phase-locked action potentials separated by 0.5 ms. This is precisely what was observed in sound sensitive vestibular afferents (FIG. 1D). Unlike the unimodal distribution of click-evoked response latencies (Zhu et al., 2011), the distributions of tone burst-evoked response latency were multimodal, providing further evidence that cycle-by-cycle vibration is responsible for organ activation. The multi-latency phase-locked distribution suggests that the tone-burst evoked activation of vestibular afferents may generate two or more positive peaks in compensatory VEMPs. We previously examined this possibility normal human subjects and found that about half of the population exhibited a second positive peak in cVEMPs (Zhang et al., 2015).

Previous studies showed that the amplitudes of VEMPs evoked by iso-intensity tone bursts depend on tone frequency (Todd et al., 2000; Lin et al., 2006; Wei et al., 2013; Ashfold et al., 2016). VEMP tuning curves have been interpreted as reflecting the resonance frequency of the vestibular end organs. For example, Todd et al. (2000) used the mass-spring damping properties of the vestibular end organs to model the VEMP tuning curves. The present FE model only includes 3D bony morphology, bulk fluid motion between the oval and round windows, and fluid compressibility and ignores wave propagation along the membranous labyrinth. But even with this simplification, results have qualitative correspondence to data (FIGS. 7A-8C), indicating that frequency tuning arises primarily from the morphology of the labyrinth as a whole, and evanescent mechanical vibration of the fluids between the oval and round windows. This mode of excitation is consistent with the fact that all vestibular organs exhibited nearly the same frequency response, but different sensitivities, at least <70 dB SL (FIG. 7C). The FE model appears to underestimate activation of the HC, AC and PC for stimuli >70 dB SL, possibly because the model does not account wave propagation from the utricule and saccule along the compliant membranous labyrinth. Propagation of vibration along the membranous labyrinth has been shown previously to be important at auditory frequencies (Iversen et al, 2018, 2019). Present experimental data also indicate a shift in tuning of the HC to auditory frequency sound as the stimulus intensity is increased. This would likely reflect a nonlinearity in the mechanics not accounted for in the present FE model.

Air-conducted sound levels used in clinical VEMP testing often is within 15-20 dB of the VEMP threshold (for review, Colebatch, 2010; Rosengren et al., 2010). As the normal human VEMP threshold is ˜70 dB above the ABR threshold (Murofushi et al., 1995), the intensity of clinical VEMP testing is about 85-90 dB above the ABR threshold. At this intensity (i.e., 80 dB SL), our previously published data showed that clicks activated a significant number of canal afferents (Zhu et al., 2011, 2014). In fact, about 66% of the AC irregular afferents were activated by the clicks and half of them exhibited strong responses (i.e., CPE>0.5). Thus, it is difficult to achieve selective activation of only otolith afferents with clicks in VEMP testing. One goal of the present study was to determine the parameters of tone bursts that allow us to achieve selective activation of the otolith afferents. Our approach was to compile tuning curves for different end organs at different intensities (FIGS. 7A-C and 8A-C). It is important to note that if tuning curves are scaled to give the same peak, the curves from all organs nearly overlap, indicating that all organs are activated by the same stimulus but to different extents. Thus, the only way to selectively excite the utricle and saccule would be to use a low strength stimulus, titrating the amplitude to avoid activation of the canals at whatever frequency is selected. For example, at 350 Hz, tone bursts primarily activated the otolith afferents. At intensity of 80 dB SL, tone bursts of 350 Hz activated 45% of otolith afferents with averaged CPE of 0.25, but they only activated 5% of canal afferents with averaged CPE of 0.05. At 1500 Hz, both canal and otolith afferents exhibited robust sound-evoked activation, but the otolith afferents can be activated at lower intensity. At 60 dB SL, tone bursts of 1500 Hz activated 50% of otolith afferents with an averaged CPE of 0.3, but only activated 7% of canal afferents with an averaged CPE of 0.02. This approach is quantitatively summarized in FIG. 12A, which plots averaged CPEs of the canal afferents and otolith afferents against intensity for two tone bursts (350 Hz: open symbols; 1500 Hz: filled symbols). By combining tone bursts of low frequency (e.g., 350 Hz) and high intensity (e.g., 80 dB SL) or high frequency (e.g., 1500 Hz) and low intensity (60 dB SL), we can achieve selectively activation of the otoliths (rectangles). In addition to selectively activating the otoliths, FIGS. 12A-B further show a novel approach to assess the canal function. FIG. 12B plots the sums of canal and otolith CPEs against tone burst intensity at two tone frequencies (350 Hz and 1500 Hz). Since the CPE-intensity slope at 350 Hz is only 0.002/dB SL for canal afferents (FIG. 12A, thin line open triangle), the summed canal and otolith CPE-intensity slope at 350 Hz is basically the otolith CPE-intensity slope. However, at 1500 Hz, the canal CPE-intensity slope is 0.01/dB SL (FIG. 12B, filled triangle), similar to the otolith CPE-intensity slope (0.011/dB SL). Thus, the summed canal and otolith CPE-intensity slope was 0.021/dB SL and 0.012/dB SL at 1500 Hz and 350 Hz, respectively, with a 1500 Hz/350 Hz slope ratio of 1.75. Thus, this 1500 Hz/350 Hz intensity slope ratio can be used as an index for canal contribution to sound-evoked vestibular responses, such as VEMPs. A ratio larger than 1 indicates significant contributions of the canal system. A ratio of near 1 indicates lack of canal contribution or deficits in the canal system.

The present invention shows that VEMP tests combining stimuli at multiple frequencies and amplitudes can provide differential diagnosis of canal and otolith inputs to compensatory vestibular circuits.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Ashford A, Huang J, Zhang C, Wei W, Mustain W, Eby T, Zhu H, Zhou W. The cervical vestibular-evoked myogenic potentials (cVEMPs) recorded along the sternocleidomastoid muscles during head rotation and flexion in normal human subjects. J Assoc Res Otolaryngol. 17(4): 303-311, 2016. doi: 10.1007/s10162-016-0566-8
2. Baird R A, Desmadryl G L, Femandez C, Goldberg J M. The vestibular nerve of the chinchilla. II. Relation between afferent response properties and peripheral innervation patterns in the semicircular canals. J. Neurophysiol. 60:182-203, 1988.
3. Broussard D M, Lisberger S G. Vestibular input to brain stem neurons that participate in motor learning in the primate vestibuloocular reflex. J. Neurophysiol. 68:1906-1909, 1992.
4. Bucks S A, Cox B C, Vlosich B A, Manning J P, Nguyen T B, Stone J S. Supporting cells remove and replace sensory receptor hair cells in a balance organ of adult mice. eLife. 6: e18128, 2017. doi: 10.7554/eLife.18128
5. Burns J, Stone J. Development and regeneration of vestibular hair cells in mammals. Semin Cell Dev Biol. 65:96-105, 2017. doi: 10.1016/j.semcdb.2016.11.001
6. Carey J P, Hirvonen T P, Hullar T E, Minor L B. Acoustic responses of vestibular afferents in a model of superior canal dehiscence. Otology & Neurotology 25:345-352, 2004.
7. Colebatch J G. Vestibular evoked potentials. Curr. Opin. in Neurol. 14:21-26, 2001.
8. Colebatch J G. Sound conclusion? Clin. Neurophysiol. 121:124-126, 2010.
9. Contini D, Holstein G R, Art J J. Synaptic cleft microenvironment influences potassium permeation and synaptic transmission in hair cells surrounded by calyx afferents in the turtle. J Physiol. 598(4): 853-889, 2020. doi: 10.1113/JP278680.
10. Cullen K E, Rey C G, Guitton D, Galiana H L. The use of system identification techniques in the analysis of oculomotor burst neuron spike train dynamics. Journal of Computational Neuroscience. 3:347-368, 1996. doi: 10.1007/BF00161093
11. Curthoys I S. A critical review of the neurophysiological evidence underlying clinical vestibular testing using sound, vibration and galvanic stimuli. Clin. Neurophysiol. 121:132-144, 2010.
12. Curthoys I S, MacDougall H G, Vidal P P, Waele C D. Sustained and transient vestibular systems: a physiological basis for interpreting vestibular function. Front Neurol. 8:117, 2017.
13. Curthoys I S, Burgess A M, Goonetilleke S C. Phase-locking of irregular guinea pig primary vestibular afferents to high frequency (>250 Hz) sound and vibration. Hear Res. 373:59-70, 2019. doi: 10.1016/j.heares.2018.12.009.
14. Curthoys I S, Kim J, McPhedran S K, Camp A J. Bone conducted vibration selectively activates irregular primary otolithic vestibular neurons in the guinea pig. Exp. Brain Res. 175(2): 256-267, 2006.
15. Curthoys I S, Vulovic V, Burgess A M, Sokolic L, Goonetilleke S C. The response of guinea pig primary utricular and saccular irregular neurons to bone-conducted vibration (BCV) and air-conducted sound (ACS). Hear Res. 331:131-143, 2016. doi: 10.1016/j.heares.2015.10.019.
16. Curthoys I S, Vulovic V, Sokolic L, Pogson J, Burgess A M. Irregular primary otolith afferents from the guinea pig utricular and saccular maculae respond to both bone conducted vibration and to air conducted sound. Brain Res. Bull. 89(1-2): 16-21, 2012.
17. Dlugaiczyk J, Gensberger K D, Straka H. Galvanic vestibular stimulation: from basic concepts to clinical applications. J Neurophysiol. 121(6): 2237-2255, 2019. doi: 10.1152/jn.00035.2019.
18. Eatock R A, Songer J E. Vestibular hair cells and afferents: two channels for head motion signals. Ann. Rev. Neurosci. 34:501-534, 2011.
19. Grieser B J, Kleiser L, and Obrist D. Identifying mechanisms behind the Tullio phenomenon: a computational study based on first principles. J Assoc Res Otolaryngol. 17(2): 103-118, 2016.
20. Goldberg J M, Fernandez C. Responses of peripheral vestibular neurons to angular and linear accelerations in the squirrel monkey. Acta Oto-laryngologica 80 (1-6): 101-110, 1975.
21. Goldberg J M, Smith C E, Fernandez C. Relation between discharge regularity and responses to externally applied galvanic currents in vestibular nerve afferents of the squirrel monkey. J. Neurophysiol. 51(6): 1236-1256, 1984.
22. Goldberg J M, Wilson V J, Cullen K E, Angelaki D E, Broussard D M, Buttnwe-Ennever J A, Fukushima K, Minor L B. Clinical manifestation of peripheral vestibular dysfunction. pp 495-524. In The Vestibular System: A Sixth Sense. Oxford University Press, 2012.
23. Hullar T E, Minor L B. High-frequency dynamics of regularly discharging canal afferents provide a linear signal for angular vestibuloocular reflexes. J. Neurophysiol. 82:2000-2005, 1999.
24 Iversen M M. Simple acoustic finite element model of rat inner ear: Input deck. Repository https://github.com/martaiversen/RatInnerEar_SimpleFEMAcousticModel, 2021.
25. Iversen M M, Rabbitt R D. Wave Mechanics of the Vestibular Semicircular Canals. Biophys J. 113(5): 1133-1149, 2017. doi: 10.1016/j.bpj.2017.08.001.
26. Iversen M M, Zhu H, Zhou W, Della Santina C C, Carey J P, Rabbitt R D. Sound abnormally stimulates the vestibular system in canal dehiscence syndrome by generating pathological fluid-mechanical waves. Scientific Reports volume 8, Article number: 10257 (2018).
27. Jiang W, Rajguru S M. Eye Movements Evoked by Pulsed Infrared Radiation of the Rat Vestibular System. Ann Biomed Eng. 46(9): 1406-1418, 2018. doi: 10.1007/s10439-018-2059-x.
28. Jombik P, Bahyl V. Short latency responses in the averaged electrooculogram elicited by vibrational impulse stimuli applied upon the skull: could they reflect function of the vestibulo-ocular reflex? J Neurol Neurosurg Psychiatry 76:222-228, 2005.
29. Lasker D M, Han G C, Park H J, Minor L B. Rotational responses of vestibular-nerve afferents innervating the semicircular canals in the C57BL/6 mouse. J. Assoc. Res. Otolaryngol. 9(3): 334-348, 2008.
30. Li L, Forge A. Morphological evidence for supporting cell to hair cell conversion in the mammalian utricular macula. International Journal of Developmental Neuroscience 15:433-446, 1997. doi: 10.1016/S0736-5748(96)00102-5
31. Lin M Y, Timmer F C A, Oriel, B S, Zhou G, Guinan J J, Kujawa S G, Herrmann B S, Merchant S N, and Rauch S D. Vestibular Evoked Myogenic Potentials (VEMP) Can Detect Asymptomatic Saccular Hydrops. Laryngoscope. 116(6): 987-992, 2006.
32. Lindeman H H. Studies of the morphology of the sensory regions of the vestibular apparatus. Erg. Anat. 42:1-113, 1969.
33. Liu S, Angelaki D E. Vestibular signals in macaque extrastriate visual cortex are functionally appropriate for heading perception. J Neurosci. (28): 8936-8945, 2009. doi: 10.1523/JNEUROSCI.1607-09.2009
34. Lysakowski A, Goldberg J M. A regional ultrastructural Analysis of the Cellular and synaptic architecture in the chinchilla cristae ampullares. J Comp Neurol. 389(3): 419-443, 1997.
35. Marlinski V, Plotnik M, Goldberg J M. Efferent actions in the chinchilla vestibular labyrinth. J Assoc Res Otolaryngol. 5(2): 126-143, 2004. doi: 10.1007/s10162-003-4029-7.
36. McCue M P, Guinan J J. Acoustically responsive fibers in the vestibular nerve of the cat. J. Neurosci. 14:6058-6070, 1994a.
37. McCue M P, Guinan J J. Influence of efferent stimulation on acoustically responsive vestibular afferents in the cat. J. Neurosci. 14:6071-6083, 1994b.
38. McCue M P, Guinan J J. Sound-evoked activity in primary afferent neurons of a mammalian vestibular system. Am. J. Otol. 18:355-360, 1997.
39. McCue M P, Guinan J J. Spontaneous activity and frequency selectivity of acoustically responsive vestibular afferents in the cat. J. Neurophysiol. 74:1563-1572, 1995.
40. Minor L B, D Solomon D, Zinreich J S, Zee D S. Sound- and/or pressure-induced vertigo due to bone dehiscence of the superior semicircular canal. Arch Otolaryngol Head Neck Surg. 124(3): 249-258, 1998.
41. Money K, Bonen L, Weaver R S. Physical properties of fluids and structures of vestibular apparatus of the pigeon. Am J. Physiol. 220(1): 140-7, 1971.
42. Murofushi T, Curthoys I S. Physiological and anatomical study of click-sensitive primary vestibular afferents in the guinea pig. Acta. Oto-Laryngologica 117:66-72, 1997.
43. Murofushi T, Curthoys I S, Topple A N, Colebatch J G, Halmagyi G M. Responses of guinea pig primary vestibular neurons to clicks. Exp. Brain Res. 103:174-178, 1995.
44 Parker D E, Tubbs R L, Littlefield V M. Visual-field displacements in human beings evoked by acoustical transients. J. Acoust. Soc. Am. 63(6): 1912-1918, 1978.
45. Rabbitt R D, Brichta A M, Tabatabaee H, Boutros P J, Ahn J, Della Santina C C, Poppi L A, Lim R. Heat pulse excitability of vestibular hair cells and afferent neurons. J Neurophysiol. 116(2): 825-843, 2016. doi: 10.1152/jn.00110.2016
46. Rajguru S M, Richter C P, Matic A I, Holstein G R, Highstein S M, Dittami G M and Richard D. Rabbitt R D. Infrared photostimulation of the crista ampullaris. J Physiol 589(6): 1283-1294, 2011.
47. Rosengren S M, Welgampola M S, Colebatch J G. Vestibular evoked myogenic potentials: Past, present and future. Clin. Neurophysiol. 121:636-651, 2010.
48. Simpson G V, Knight R T, Brailowsky S, Prospero-Garcia O, Scabini D. Altered peripheral and brainstem auditory function in aged rats. Brain Res. 348:28-35, 1985.
49. Songer J E, Eatock R A. Tuning and timing in mammalian type I hair cells and calyceal synapses. J Neurosci. 33(8): 3706-3724, 2013. doi: 10.1523/JNEUROSCI.4067-12.2013.
50. Spoon C, Grant W. Biomechanics of hair cell kinocilia: experimental measurement of kinocilium shaft stiffness and base rotational stiffness with Euler-Bernoulli and Timoshenko beam analysis. J Exp Biol. 214(5): 862-870, 2011. doi: 10.1242/jeb.051151
51. Steer R W, Yao T L, Young L R, Meiry J L. Physical properties of labyrinthine fluids and quantification of the phenomenon of caloric stimulation. Third symposium on the role of the vestibular organs in space exploration. Pensacola, FL Jan. 23-27, 1967.
52. Tonndorf J, Tabor J R. Closure of the cochlear windows: its effect upon air- and bone-conduction. Ann Otol Rhinol Laryngol 71:5-29, 1962.
53. Todd NPM, Cody F W, Banks J R. A saccular origin of frequency tuning in myogenic vestibular evoked potentials?: implications for human responses to loud sounds. Hear Res. 141(1-2): 180-188, 2000. doi: 10.1016/s0378-5955(99)00222-1.
54. Todd NPM, Rosengren S M, Aw S T, Colebatch J G. Ocular vestibular evoked myogenic potentials (OVEMPs) produced by air- and bone-conducted sound. Clin Neurophysiol 118:381-90, 2007.
55. Tullio P. Das Ohr und die Entstehung der Sprache und Schrift. Berlin: Urban & Schwarzenberg, 1929.
56. Uchino Y, Kushiro K. Differences between otolith- and semicircular canal-activated neural circuitry in the vestibular system. Neurosci Res. 71(4): 315-327, 2011.
57. Uchino Y, Sasaki M, Sato H, Bai R, Kawamoto E. Otolith and canal integration on single vestibular neurons in cats. Exp. Brain Res. 164:271-285, 2005.
58. Ward B K, Roberts D C, Otero-Millan J, Zee D S. A decade of magnetic vestibular stimulation: from serendipity to physics to the clinic. J Neurophysiol. 121(6): 2013-2019, 2019. doi: 10.1152/jn.00873.2018.
59. Wei W, Jeffcoat B, Mustain W, Xu Y, Eby T, Zhu H, Tang X, Zhou W. Vestibular-evoked myogenic potentials (VEMP) recorded from different sites of the sternocleidomastoid muscles in normal human subjects. J. Assoc. Res. Otolaryngol. 14(1): 37-47, 2013.
60. Wilson V J, Schor R H. The neural substrate of the vestibulocollic reflex: What needs to be learned? Exp. Brain Res. 129:483-493, 1999.
61. Wit H P, Bleeker J D, Mulder H H. Responses of pigeon vestibular nerve fibers to sound and vibration with audio frequencies. J. Acoust. Soc. Am. 75(1): 202-208, 1984.
62. Xu Y, Simpson I, Tang X, Zhou W. Acoustic clicks activate both the canal and otolith vestibulo-ocular reflex pathways in behaving monkeys. J. Assoc. Res. Otolaryngol. 10 (4): 569-577, 2009.
63. Young E D, Fernandez C, Goldberg J M. Responses of squirrel monkey vestibular neurons to audio-frequency sound and head vibration. Acta Otolaryngol. 84:352-360, 1977.
64. Zhang C M, Huang J, Xu Y, Mustain W, Zhu H, Zhou W. Low frequency tone burst evokes a new positive peak (P2) in cervical VEMPs between the traditional P1-N1. ARO, 39:286, 2016.
65. Zhu H, Tang X, Wei W, Maklad A, Mustain W, Rabbitt R, Highstein S, Allison J, Zhou W. Input-output functions of vestibular afferent responses to air-conducted clicks in rats. JARO. 15:73-86, 2014.
66. Zhu H, Tang X, Wei W, Xu Y, Mustain W, Zhou W, Click-evoked responses in vestibular afferents in rats. J. Neurophysiol. 106(2): 754-763, 2011.
67. Zhou W, Weldon P, Tang B, King W M. Rapid Motor Learning in the Translational Vestibulo-Ocular Reflex. J Neurosci. 23(10): 4288-4298, 2003. doi: 10.1523/JNEUROSCI.23-10-04288.2003
68. Zhou W, Xu Y, Simpson I, Cai Y D. Multiplicative computation in the vestibulo-ocular reflex (VOR). J. Neurophysiol. 97:2780-2789, 2007.

It will be understood that various details of the presently disclosed subject matter can be changed without departing from the scope of the subject matter disclosed herein. Furthermore, the foregoing description is for the purpose of illustration only, and not for the purpose of limitation.

1. A method of discriminative vestibular-evoked myogenic potential testing (VEMP) in a subject in need thereof, comprising:
providing a first acoustic stimuli to the subject to activate otolith afferents to provide a response, the first acoustic stimuli being at a first predetermined frequency and a first set predetermined intensities;
providing a second acoustic stimuli to the subject to activate both canal and otolith afferents to provide a response, the second acoustic stimuli being at a second predetermined frequency and a second set of predetermined intensities;
determining and comparing the responses of the first and second acoustic stimuli to assess canal and otolith contributions to sound-evoked responses of the vestibular end organs.

2. The method of claim 1, wherein the first and second acoustic stimuli are series of at least three tone bursts.

3. The method of claim 1, wherein the first predetermined frequency is below 500 Hz and the first set of predetermined intensities is a series of tone bursts up to about 100 dB HL.

4. The method of claim 3, wherein the series of tone bursts are at least four tone bursts in intensity increments of 10 dB HL.

5. The method of claim 1, wherein the series of tone bursts intensity are in increments of about 10 dB HL.

6. The method of claim 1, wherein the first acoustic stimuli is a series of at least four tone bursts at a frequency below 500 Hz and the intensity up to about 100 dB HL.

7. The method of claim 3, wherein the tone bursts are at increasing intensities up to about 100 dB HL.

8. The method of claim 1, wherein the second predetermined frequency is about 1500 Hz and the second intensity is up to 100 dB HL.

9. The method of claim 1, wherein the second acoustic stimuli is a series of tone bursts at a frequency of about 1500 Hz and the intensity of up to about 100 dB HL.

10. The method of claim 9, wherein the tone bursts are at increasing intensities up to about 100 dB HL.

11. The method of claim 10, wherein there are at least four tone bursts of different intensities.

12. The method of claim 6, wherein the first acoustic stimuli discriminately activates otolith afferents.

13. The method of claim 1, where in the activation is measured by the peaks of VEMP responses.

14. The method of claim 13, where the peaks of the VEMP responses are plotted to form an intensity-amplitude curve.

15. The method of claim 1, wherein the first predetermined frequency is 350 Hz or below.

16. The method of claim 14, wherein intensity and VEMP amplitude curves of the high frequency and low frequency are compared to obtain a ratio of the slopes of the two intensity-amplitude curves.

17. A method of discriminative sound-evoked vestibular myogenic potential testing (VEMP) in a subject in need thereof, comprising:
providing a first acoustic stimuli to the subject to activate otolith afferents to provide a response, the first acoustic stimuli being at a first predetermined frequency and a first set predetermined intensities;
providing a second acoustic stimuli to the subject to activate otolith afferents to provide a response, the second acoustic stimuli being at a second predetermined frequency and a second set predetermined intensities;
determining and comparing the response of the first and second acoustic stimuli to assess otolith and canal function contributions to sound-evoked responses of the vestibular end organs.

18. The method of claim 17, wherein the first and second acoustic stimuli are series of at least three tone bursts, and wherein the series of tone bursts are at least four tone bursts in increasing increments of about 10 to 30 dB HL.

19. The method of claim 17, wherein the first and second predetermined frequencies are below 500 Hz and the first and second set of predetermined intensities are a series of tone bursts up to about 100 dB HL.

20. (canceled)

21. The method of claim 17, wherein the first and second acoustic stimuli are series of at least four tone bursts at frequency below 350 Hz.
